(19) United States
(12) Patent Application Publication
(10) Pub. No.: US 2005/0064524 A1
(43) Pub. Date: Mar. 24, 2005

(54) Population of cells utilizable for substance detection and methods and devices using same

(76) Inventors: Mordechai Deutsch, Doar Na Lev-HaSharon (IL); Naomi Zurgil, Herzlia (IL)

Correspondence Address: Martin Moynihan;c/o ANTHONY CASTORINA, SUITE 207, 2001 JEFFERSON DAVIS HIGHWAY, ARLINGTON, VA, 22202, US

(21) Appl. No.: 10/916,380

(22) Filed: Aug. 11, 2004

Related U.S. Application Data
(60) Provisional application No. 60/493,813, filed on Aug. 11, 2003.

(51) Int. Cl. G01N 33/574; C12N 5/08

(52) U.S. Cl. 435/7.23; 435/366

(57) ABSTRACT

An isolated population of cells is provided. The isolated population of cells comprising at least one secretor cell capable of secreting a molecule and at least one sensor cell capable of producing a detectable signal upon being exposed to the molecule.

This application claims the benefit of priority of U.S. provisional patent application No. 60/493,813, filed Aug. 11, 2003, which is hereby incorporated by reference.

### FIELD AND BACKGROUND OF THE INVENTION

The present invention relates to novel populations of cells and, more particularly, to methods of using such populations of cells for detection of substances.

The ability to qualify and quantify substances present in a liquid or gaseous samples is of great importance in clinical, environmental, health and safety, remote sensing, military, food/beverage and chemical processing applications.

There are two general approaches for analyte (i.e., substance) detection. Traditional approaches are based on chemical or physical analysis allowing highly accurate and sensitive determination of the exact composition of any sample [e.g., liquid chromatography (LC), gas chromatography (GC), and supercritical fluid chromatography (SFC)]. However, these techniques are time consuming, extremely expensive, require sample preconcentration, and are difficult or impossible to adapt to field use. In addition, such technologies fail to provide data as to the bioavailability of pollutants, their effects on living systems, and their synergistic/antagonistic behavior in mixtures.

A biosensor is a device that qualifies and/or quantifies a physiological or biochemical signal. Biosensors have been developed to overcome some of the shortcomings of the classical analyte detection techniques. Good biosensing systems are characterized by specificity, sensitivity, reliability, portability, ability to function even in optically opaque solutions, real-time analysis and simplicity of operation. Biosensors couple a biological component with an electronic transducer and thus enable conversion of a biochemical signal into a quantifiable electrical response.

The use of whole cells as the biosensing element negates the lengthy procedure of enzyme purifications, preserves the enzymes in their natural environment and protects it from inactivation by external toxicants such as heavy metals. Whole cells also provide a multipurpose catalyst especially when the process requires the participation of a number of enzymes in sequence. Whole cells have been used either in viable or non-viable form. Viable microbes, for example, can metabolize various organic compounds resulting in various end products like ammonia, carbon dioxide, acids and the like, which can be monitored using a variety of transducers [Burlage (1994) Annu. Rev. Microbiol. 48: 291-309; Riedel (1998) Anal. Lett. 31:1-12; Arikawa (1998) Mulchandani, Rogers (Eds.) Enzyme and Microbial Biosensors: Techniques and Protocols. Humanae Press, Totowa, N.J., pp.225-235; and Simonian (1998) Mulchandani, Rogers (Eds.) Enzyme and Microbial Biosensors: Techniques and Protocols. Humanae Press, Totowa, N.J. pp:237-248].

While reducing the present invention to practice, the present inventors designed novel approaches for substance detection and cellular classification using cellular biosensors.

### SUMMARY OF THE INVENTION

According to one aspect of the present invention there is provided an isolated population of cells comprising at least one secretor cell capable of secreting a molecule and at least one sensor cell capable of producing a detectable signal upon being exposed to the molecule.

According to another aspect of the present invention there is provided a method of detecting presence, absence or level of a substance in a sample, the method comprising: (a) exposing at least one secretor cell to the sample, the at least one secretor cell being capable of secreting a molecule when exposed to the substance in the sample; (b) exposing at least one sensor cell to the molecule, the at least one sensor cell being capable of producing a detectable signal when exposed to the molecule; and (c) analyzing the detectable signal to thereby detect presence, absence or level of the substance in the sample.

According to yet another aspect of the present invention there is provided a method of identifying cells expressing a molecule of interest, the method comprising exposing sensor cells to a plurality of cells potentially capable of secreting the molecule of interest, the sensor cells being capable of producing a detectable signal when exposed to the molecule of interest, thereby identifying the cells expressing the molecule of interest.

According to further features in preferred embodiments of the invention described below, the molecule is selected from the group consisting of a small molecule chemical, an ion, a carbohydrate and a polypeptide.

According to still further features in the described preferred embodiments the small molecule chemical is selected from the group consisting of a reactive oxygen species and a reactive nitrogen species.

According to still further features in the described preferred embodiments the ion is selected from the group consisting of calcium, magnesium, zinc and phosphate.

According to still further features in the described preferred embodiments the polypeptide is selected from the group consisting of a growth factor, a hormone, a coagulating factor, a cytokine and a chemokine.

According to still further features in the described preferred embodiments the secretor cell is a cancer cell.

According to still further features in the described preferred embodiments the population of cells is attached to a support and whereas each of the at least one secretor cell and the at least one sensor cell is attached to the support in an addressable manner. According to still further features in the described preferred embodiments the support is configured as a microscope slide.

According to still further features in the described preferred embodiments the support is configured as a multiwell plate.

According to still further features in the described preferred embodiments the at least one secretor cell and the at least ore sensor cell are in fluid communication therebetween on the support.

According to still further features in the described preferred embodiments each well of the multiwell plate has a volume between 1×10⁻⁵-1×10⁻¹⁵ μL.

According to still further features in the described preferred embodiments the at least one secretor cell and the at least one sensor cell are eukaryotic cells.

According to still further features in the described preferred embodiments the at least one secretor cell and the at least one sensor cell are prokaryotic cells.

According to still further features in the described preferred embodiments the detectable signal is selected from the group consisting of a morphological signal, a fluorogenic signal and a chromogenic signal.

The present invention successfully addresses the shortcomings of the presently known configurations by providing novel populations of cells which can be used for detection of substances in a sample.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 describes various stress conditions and agents which cause oxidative stress as well as molecular damage and cellular effects thereof.

FIG. 2 is a scheme illustrating pathways leading to the generation of ROS/RNS. RNS generation—NO is synthesized in several cell types by NO synthase (NOS). NOS converts L-arginine to L-citrulline and NO. There are at least three different families of NOS. Two are constitutively expressed including NOS type I (NNOS—neuronal NOS) and NOS type III (eNOS—endothelial NOS). Their activities are regulated by the level of intracellular calcium and are thought to constitutively synthesize NO for intercellular signaling and vasoregulation. The third variant, NOS type II, is an inducible NOS (iNOS) expressed following inflammatory stimulation in a number of cell types, like macrophages, chondrocytes, neutrophils, hepatocytes, epithelium and smooth muscle cells. White blood cells respond to infection with an increased consumption of oxygen, referred to as the respiratory burst. The overall result of this process is reduction of oxygen to superoxide (O₂), which is performed by the nicotinamide adenine dinucleotide phosphate oxidase (NADPH-oxidase) enzyme system. O₂⁻ is released to the phagosome and/or to the extracellular compartment. Superoxide and nitric oxide (NO.) react with each other at a near diffusion-limited rate to form peroxynitrite (ONOO—), which is a potent oxidant. ROS generation—Superoxide (O₂) anion is metabolized via the dismutation reaction 2O₂⁻+2H⁺O₂+H₂O₂, which is catalyzed by superoxide oxidoreductase dismutase (SOD), a cytoplasmic enzyme that is constitutively expressed and a mitochondrial enzyme which is induced in response to oxidant stress. The H₂O₂ produced by the dismutation of O₂⁻ is converted by one pathway to H₂O and O₂ by catalase (CAT) in peroxisomes and by glutathione peroxidase (GSH-PX) in the cytoplasm, at the expense of reduced glutathione (GSH), leading to the formation of oxidized glutathione disulphide (GSSG) that is recycled back to GSH by glutathione reductase (GSSGRD). H₂O₂ could be further converted by another pathway involving iron into hydroxyl radical (OH), an injurious ROS causing cellular damage. This iron-catalyzed reaction, known as the Fenton-like reaction, is impeded by the iron chelator desferrioxamine (DSF), which is also capable of neutralizing the toxicity of OH.

FIG. 3 is a graph depicting NO generation by DETA/NO as a function of time.

FIGS. 4a-b are graphs depicting dose response of NO generation by DETA/NO.

FIG. 5 is a bar graph depicting fluorescent intensity (FI) values of U937 cells labeled with DA-2FDA and incubated in the absence or presence of NO donor or in the presence of medium pre-incubated with DETA/NO.

FIGS. 6a-b are light (FIG. 6a) and fluorescence (FIG. 6b) photomicrographs depicting individual U937 cells stained with DAF-2DA and incubated for 1 hour in the presence of DETA/NO.

FIGS. 7a-b are graphs depicting intracellular NO levels in individual living cells as measured using a DAF-2DA probe.

FIGS. 8a-b are graphs depicting time dependence of Fl and Fluorescence Polarization (FP) measured in two individual cells.

FIG. 9 is a graph depicting distribution patterns of individual U937 cells labeled with DA-2FDA in the presence and absence of an NO donor.

FIG. 10 is a graph depicting changes in Fl values upon incubation of U937 cells including a DAF-2A probe with varying concentrations of DETA/NO.

FIG. 11 is a scheme illustrating subcellular distribution of three ROS probes and their oxidized fluorescent species.

FIG. 12 is scheme illustrating the mechanism of action of the dihydrorhodamine 123 probe.

FIG. 13 is a graph depicting quantitative measurements of intracellular ROS concentration in individual living cells by Dihydro rhodamine 123 (DHR123).

FIGS. 14a-b are graphs depicting measurements of intracellular ROS concentration in a group of individual cells.

FIGS. 15a-b are graphs depicting measurements of intracellular ROS concentrations in two representative individual cells.

FIGS. 16a-b are graphs depicting kinetics of FI and FP of DHR stained individual U937 cells following stimulation with hydrogen peroxide.

FIG. 17 is a graph depicting the effect of various concentrations of hydrogen peroxide on the rate of FI change. Cells were preloaded with DHR and then exposed to hydrogen peroxide for 10 minutes. The mean rate of FI change with time was measured.

FIG. 18 is scheme illustrating the mechanism of action of the Dichlorodihydrofluorescein diacetate (DCFH-DA) probe.

FIG. 19 is a graph depicting intracellular oxidative activity (ROS) measurements in individual living cells by DCFH-DA.

FIG. 20 is scheme illustrating the mechanism of action of the Dihydroethidium (DHE) probe.

FIGS. 21a-b are light (FIG. 6a) and fluorescence (FIG. 6b) photomicrographs of individual U937 cells stained with DHE and incubated for 1 hour in the presence of 50 μM hydrogen peroxide.

FIGS. 22a-b are graphs depicting specificity of NOS (FIG. 22a) and ROS (FIG. 22b) fluorescent probes to respective donors. Diamonds denote control; Squares denote hydrogen peroxide; circles denote DETA/NO.

FIGS. 23a-b are photomicrographs depicting temporal onset of ROS generation in different intracellular locations of an individual THP1 cells double stained with DHR123 and DHE.

FIG. 24 is a graph depicting fluorescence zone size (dashed lines, triangles) and average FI (solid lines, diamonds) generated by the different ROS probes.

FIGS. 25a-b are graphs depicting endogenous ROS generation upon exposure of stained cells to lysophosphatidylecholine (LPC). Time dependent ROS production is shown in FIG. 25a. Dose dependent ROS production is shown in FIG. 25b.

FIGS. 26a-b are graphs depicting intracellular ROS levels (FIG. 26a) and mitochondrial membrane potential (FIG. 26b) in individual living cells exposed to hydrogen peroxide.

FIG. 27 is a graph depicting temporal relationship between kinetic of ROS generation (indicated by DHR, circles) and the onset of changes in mitochondrial membrane potential (indicated by TMRM, diamonds) in cells exposed to hydrogen peroxide stimulus.

FIGS. 28a-b are graphs depicting simultaneous measurements of NO and ROS in individual living cells probed with DAF-2DA and DHE following exposure to hydrogen peroxide and DETA/NO. FIG. 28a—addition of hydrogen peroxide to DETA/NO treated cells. FIG. 28b—addition of DETA/NO to hydrogen peroxide treated cells.

FIGS. 29a-b show ratiometric measurements of fluorescent intensity ratio (FIR). The ratio between FI(NO) and Fl (ROS), were used for the simultaneous monitoring of ROS and NOS rates of formation in individual live cells. An increase in FIR occurred when the rate of NO production exceeded the rate of ROS formation (FIG. 29a) and decreased as the rate of ROS formation exceeded that of NO (FIG. 29b).

FIGS. 30a-c are photographs depicting time dependent endogenous ROS generation in sensor cells upon exposure to ROS secreting cells. FIG. 30a shows sensor cells stained with DHR. FIG. 30b shows 1 minute coincubation of DHR stained sensor cells with ROS donating cells (secreting cells. FIG. 30c shows 10 minutes co-incubation of DHR stained sensor cells with ROS donating cells (secreting cells.

FIGS. 31a-b are photographs depicting experimental controls for the assay described in FIGS. 31a-c, above. FIG. 31a shows untreated DHR stained cells (sensors) following addition of unstained untreated cells. FIGS. 3 lb shows untreated DHR stained cells (sensors) following addition of stained untreated cells FIGS. 32a-c are schematic illustrations depicting a general configuration of the device of the present invention.

### DESCRIPTION OF THE PREFERRED EMBODIMENTS

The present invention is of novel populations of cells which can be utilized for cell classification and substance identification.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Biosensors are fast becoming the preferred approach for analyte detection in cases where rapid qualification and/or quantification of substances present in a liquid or gaseous samples are desired. Numerous examples of biosensors exist in the art including enzyme-based biosensors, antibody-based biosensors and whole cell-based biosensors.

While reducing the present invention to practice, the present inventors have devised a novel approach for analyte identification, cell classification and high throughput screening of drugs using a dedicated pairedcell approach.

As is illustrated in the Examples section, which follows, the paired cell approach of the present invention could provide realtime accurate measurement of reactive oxygen and nitrogen species produced in a cell following stimulation. It will be appreciated that realtime measurement of such gaseous metabolites is not trivial due to their extremely short half-life and heterogeneity of production. Thus, the present invention allows for the first time to measure the secretion of molecules, which have thusfar eluded accurate real time detection.

Thus, according to one aspect of the present invention there is provided an isolated population of cells composed of at least one secretor cell capable of secreting a molecule and at least one sensor cell capable of producing a detectable signal upon being exposed to the molecule.

As used herein “an isolated population of cells” refers to prokaryotic or eukaryotic cell isolates of natural (e.g., isolated from a tissue, a host, the environment etc) or recombinant (e.g., isolated from transformed populations) origin.

Examples of prokaryotic cells which can be used in accordance with this aspect of the present invention include but are not limited to bacterial cells, such as Pseudomonas, Bacillus, Bacteriodes, Vibrio, Yersinia, Clostridium, Mycobacterium, Mycoplasma, Coryynebacterium, Escherichia, Salmonella, Shigella, Rhodococcus, Methanococcus, Micrococcus, Arthrobacter, Listeria, Klebsiella, Aeromonas, Streptomyces and Xanthomonas.

Examples of eukaryotic cells which can be used in accordance with this aspect of the present invention include but are not limited to cell-lines, primary cultures or permanent cell cultures of fungal cells such as Aspergillus niger and Ustilago maydis [Regenfelder, E. et al. (1997) EMBO J. 16:1934-1942], yeast cells (see U.S. Pat. Nos. 5,691,188, 5,482,835), such as Saccharomyces, Pichia, Zygosaccharomyces, Trichoderma, Candida, and Hansenula, plant cells, insect cells, nematoda cells such as c. elegans, invertebrate cells, vetebrate cells and mammalian cells such as fibroblasts, epithelial cells, endothelial cells, lymphoid cells, neuronal cells and the like. Such cells are commercially availa ble from the American Type Culture Co. (Rockville, Md., USA).

Each of the secretor cell or sensor cell of the above described cell population can be a normal cell or a cell which originated from a disease state, such as cancer.

The molecule or molecules which are secreted from the secretor cells of the present invention and subsequently detected by the sensor cell of the present invention may be a molecule naturally expressed and secreted by the secretor cell (endogenous thereto) or it may be a molecule which is not naturally secreted by the secretor cell (exogenous thereto). In any case, secretion can be a native function of the secreted molecule (e.g, the molecule natively includes a secretion signal sequence) or in turn, the molecule can be modified to enable secretion thereof from the cell. For example, in cases where the molecule is a protein, it can be genetically manipulated to include a signal peptide which directs the secretion of proteins from cells; and/or to leaving out hydrophobic patches which normally lead to insertion of the protein in membranes. Alternatively, secretor cells may be mildly permeabilized to passively secrete the molecule. Methods of membrane permeabilization are described in details in Ojcius C. Res. Immunol. (1996)177(3):175-88.

Examples of molecules, which may be secreted by the secretor cells of the present invention, include, but are not limited to, small molecule chemicals (e.g., reactive oxygen and/or nitrogen species, see Examples section which follows), ions (e.g., calcium), carbohydrates (e.g., heparin), polynucleotides and polypeptides (e.g., growth factors, hormones, coagulating factors and secreted enzymes).

As mentioned hereinabove, sensor cells of this aspect of the present invention are selected capable of producing a detectable signal upon being exposed to the molecule secreted from the secretor cell.

As used herein the phrase “detectable signal” refers to any cellular indication which can be visualized and, preferably, measured.

The detectable signal according to this aspect of the present invention can be a morphological signal, wherein the morphology of the sensor cell is altered upon exposure to the molecule. A morphological signal may include the formation of dynamic actin-based structures such as, lamellopodia and buds, changes in cell polarity, re-organization of cytoskelleton and organelles and changes in organelle structure (i.e., enlarged or reduced size) or number. Visualization of such morphological signals may be facilitated by specific dyes and/or a magnifying optical device, such as a fluorescent microscope, a con-focal microscope and an electron microscope.

Alternatively, the detectable signal can be a viability signal, wherein the viability of the sensor cell is reduced (e.g., apoptoss, senescence) or enhanced (e.g., proliferation) upon exposure to the molecule.

Yet alternatively, the detectable signal can be a biochemical signal, wherein the activity or expression of an enzyme or an enzymatic pathway is altered upon exposure to the molecule.

Biochemical, viability and/or morphological change signals may be enhanced/visualized using antibodies, dyes or reporter expression constructs, which provide a chromogenic, fluorogenic or morphological signal.

Examples of dyes, include but are not limited to, subcellular organelles and structures stains, lipid stains such as fluorescent analogs for natural lipids (e.g., phospholipids, sphingolipids, fatty acids, triglycerides and steroids), probes for detecting various reactive oxygen species (such as hydroperoxides in living cells membranes) and fluorescent indicators for ion detection such as, magnesium, sodium, potassium, hydrogen, zinc, chloride protons etc. Such dyes are well known in the art and are commercially available from for example, molecular probes, [for example see, “Handbook of Fluorescent Probes and Research Chemicals” (www.molecularprobes.com/handbook/sections/1200.html), Chapter 11—Probes for Actin, Tubulin and Nucleotide-Binding Proteins, Chapter 12—Probes for Organelles, Chapter 13—Probes for Lipids and Membranes, Chapter 18—Probes for Signal Transduction, Chapter 19—Probes for Reactive Oxygen Species, Including Nitric Oxide, Chapter 20—Indicators for Ca²⁺, Mg²⁺, Zn²⁺ and Other Metals, Chapter 21—pH Indicators].

As mentioned, the sensor cell may include a reporter expression construct, which expresses a detectable reporter molecule when the cell is exposed to the molecule.

As used herein “reporter expression construct” refers to a vector which includes a polynucle otide sequence encoding a reporter. Preferably, the polynucleotide sequence is positioned in the construct under the transcriptional control of at least one cis-regulatory element suitable for directing transcription in the sensor cell upon exposure to the molecule.

As used herein a “cis acting regulatory element” refers to a naturally occurring or artificial polynucleotide sequence, which binds a trans acting regulator and regulates the transcription of a coding sequence located downstream thereto. For example, a transcriptional regulatory element can be at least a part of a promoter sequence which is activated by a specific transcriptional regulator or it can be an enhancer which can be adjacent or distant to a promoter sequence and which functions in up regulating the transcription therefrom.

The cis-acting regulatory element of this aspect of the present invention can be regulated directly or indirectly by the molecule which is secreted from the secretor cells of the present invention. The cis-acting regulatory element may be a stress-regulated promoter, which is activated in response to cellular stress produced by exposure of the cell to, for example, chemicals, ions, heavy metals, changes in temperature, changes in pH, as well as agents producing oxidative damage (e.g., ROS), DNA damage, anaerobiosis, and changes in nitrate availability or pathogenesis.

Examples of ROS inducible promoters include, but are not limited to, the vitamin D3 regulated protein (VDUP1) promoter [Kim (2004) Biochem. Biophys. Res. Commun. 315(2):369-75] and the ofos promoter [Cheng (1999) Cardiovasc. Res. 41 :654-62].

A cis acting regulatory element can also be a translational regulatory sequence element in which case such a sequence can bind a translational regulator, whichup regulates translation.

The term “expression” refers to the biosynthesis of a gene product. For example, in the case of the reporter polypeptide, expression involves the transcription of the reporter gene into messenger RNA (mRNA) and the translation of the mRNA into one or more polypeptides.

As used herein “reporter polypeptide” refers to a polypeptide gene product, which, can be quantitated either directly or indirectly. For example, a reporter polypeptide can be an enzyme which when in the preserce of a suitable substrate generates chromogenic products. Such enzymes include but are not limited to alkaline phosphatase, β-galactosidase, β-D-glucoronidase (GUS), luciferase and the like. A reporter polypeptide can also be a fluorescer such as the polypeptides belonging to the green fluorescent protein family including the green fluorescent protein, the yellow fluorescent Frotein, the cyan fluorescent protein and the red fluorescent protein as well as their enhanced derivatives. In such a case, the reporter polypeptide can be quantified via its fluorescence, which is generated upon the application of a suitable excitatory light. Alternatively, a polypeptide label can be an epitope tag, a fairly unique polypeptide sequence to which a specific antibody can bind without substantially cross reacting with other cellular epitopes. Such epitope tags include a Myc tag, a Flag tag, a His tag, a Leucine tag, an IgG tag, a streptavidin tag and the like. Further details on reporter polypeptides can be found in Misawa et al. (2000) PNAS 97:3062-3066.

The reporter expression construct can be introduced into the cell using a variety of molecular and biochemical methods known in the art. Examples include, but are not limited to, transfection, conjugation, electroporation, calcium phosphate-precipitation, direct microinjection, liposome fusion, viral infection and the like. Selection of a suitable introduction method is dependent upon the host cell and the type of construct used.

The above described cell population can be employed in a variety of applications. For example, in the environmental field, the cell population of the present invention can be employed to detect the presence of pollutants such as halogenated hydrocarbons (used as pesticides), polycyclic aromatic hydrocarbons (carcinogenic compounds), acrylamide, acrylic acid and acrylonitrile, organophosphorous compounds (used as pesticides, insecticides, and chemical warfare agents), nitroaromatic compounds, such as nitrophenols, picric acid, trinitrotoluene (used as xenobiotics present in wastes of chemical armament plants as in civil factories for dye, pesticide, and other chemical manufacturing). Alternatively, the cell population of the present invention can be employed in the food and fermentation industries, where there is a need for quick and specific analytical tools. Analysis is needed for monitoring nutritional parameters, food additives, food contaminants, microbial counts, shelf life assessment, compliance with specifications or regulations, and other olfactory properties like smell and odor. In pharmaceuticals and medicine, the cell population of the present invertion can be used for drug identification and qualification (e.g., determination of active ingredients in pharmaceutical formulations]. The cell populations of the present invention can also be used for detecting narcotics and explosives such as trinitrotoluene (TNT), cyclonite (RDX), pentaerythritol tetranitrate (PETN) C-4 class explosives, and combinations thereof [Yinon, Y. and Zitrin, S. (1993) Modern Methods and Applications in Analysis of Explosives, John Wiley & Sons, Ltd., Sussex, U. K.].

Thus, according to another aspect of the present invention there is provided a method of detecting presence, absence or level of a substance in a sample.

As used herein the term “substance” refers to a molecule or a mixture of molecules in a liquid, gaseous or aerosol medium. Examples of substances include, but are not limited to, small molecules such as naturally occurring compounds (e.g., compounds derived from plant extracts, microbial broths, and the like) or synthetic compounds having molecular weights of less than about 10,000 daltons, preferably less than about 5,000 daltons, and most preferably less than about 1,500 daltons, ions (e.g., electrolytes and metals), polypeptides, polynucleotides, peptides, nucleotides, carbohydrates, fatty acids, steroids and the like. Substances typically include at least one functional group necessary for biological interactions (e.g., amine group, carbonyl group, hydroxyl group, carboxyl group).

As used herein the term “sample” refers to any liquid, gaseous or aerosol medium. When needed, the sample is diluted into a biocompatible medium which allows cell maintenance and/or expansion therein.

The method is effected by exposing the sample to at least one secretor cell being capable of secreting a molecule when exposed to the substance in the sample and exposing at least one sensor cell to the molecule such that a detectable signal is produced as described above, and analyzing the detectable signal to thereby detect presence, absence or level of the substance in the sample.

It will be appreciated that the sample can be either contacted with or introduced into the secretor cell, using molecular or biochemical methodologies well known in the art. Examples include but are not limited to, transfection, conjugation, electroporation, calcium phosphate-precipitation, direct microinjection, liposome fusion and the like.

A number of controls may be included in the above-described methodology For example, sensor cells which are designed to constitutively express the reporter polypeptide, are preferably included for qualifying the reagents used. Alternatively, or additionally, naive sensor cells, not including the reporter polynucleotide encoding the reporter polypeptide or dye, may be included for monitoring background signal.

Analysis of the detectable signal produced by the sensor cells of the present invention may be effected using, a magnifying optical device, typically equipped with filters for detection of the detectable signal, such as that produced by the reporter polypeptide or dye, described hereinabove. When needed, signal amplification can be effected using a photoamplifier.

Analysis of measurement data is preferably effected using an imaging software.

To simplify analysis, especially when applied for high throughput greening (e.g., screening multiple samples), sensor cells and secretor cells are attached to a support in an addressable manner, thereby enabling signal identification of each discrete population of sensor and secretor cells.

Referring now to the drawings, FIGS. 32a-c illustrate a device for detecting presence, absence or level of a substance in a sample which is referred to herein as device 10.

Device 10 includes a support 101 configured for supporting sensor cells 108 and secretor cells 110 of the present invention, in an addressable manner, i.e., enabling identification of discrete groups of paired secretor cells and sensor cells on the array. Support 101 is preferably fabricated from a material, which can accommodate discrete individual sites (e.g, wells, chambers, etc.) configured for containment, attachment or association of sensor cells 108 and secretor cells 110. Examples of materials suitable for fabrication of support 101 include, but are not limited to glass (including modified or functiomlized glass), plastics (e.g., acrylics, polystyrene, polypropylene, polyethylene, polybutylene, polyurethanes), polysaccharides, nylon, nitrocellulose, resins, silica, silica-based materials (e.g., silicon), carbon, metals, inorganic glasses, optical fiber bundles (see U.S. Pat. No. 6,377,721) and the like. Support 101 is preferably selected such that it allows optical detection of a signal generated by the sensor cells 108 contained therein or attached thereto. Support 101 is typically planar, although other configurations can also be used in device 10. For example, three dimensional configurations of support 101, can be generated by embedding cells in a porous block of plastic that allows detection of a signal generated by these cells. Similarly, sensor cells 108 and secretor cells 110 can be placed on the inside surface of a tube, for flow through sample analysis to minimize sample volume.

At least one surface 103 of support 101 is fabricated with, or is modified (e.g., etched) to include discrete locations (e.g., chambers, wells) 102 which are configured or modified so as to enable holding one or more secretor cells 110 and sensor cells 108.

Locations 102 may be regularly or randomly distributed in or on surface 103. A preferred embodiment utilizes a regular pattern of locations such that the sites may be addressed using an X-Y coordinate system.

In a preferred embodiment, locations 102 are formed as microwells 107, i.e. depressions in the surface of the support (i.e., multiwell plate). Such a multiwell plate can be configured as a standard multiwell microtiter plate. These plates include 6n wells arranged in a rectangular packing. Commercially available microtiter plates include 6, 24, 96, 384 or 1536 wells and can be obtained from Nalge Nunc (Rochester, N.Y.). It will be appreciated that a single multiwell plate may be composed from a number of materials. For example, surfaces, which are in contact with the cells, may be made of biocompatible, preferably adhesive materials while other surfaces, such as interwell surfaces, may be made of other materials.

The walls of wells of the multiwell plate may be integrally formed with the bottom surface of the wells. Alternatively, the multiwell plate of this aspect of the present invention may include at bast one distinct well-wall component attached to the bottom surface.

The size and volume of each well of the multiwell plates of this aspect of the present invention depends on the type (eukaryotic vs. prokaryotic cells) and number of cells used. For example, picowell plates may be employed when one or several cells are included in each location 102.

A number of picowell supports are known in the art. See for example, Mrksich and Whitesides (1996) Ann. Rev. Biophys. Biomol. Struct. 25:55-78; Craighead et al. (1982) J. Vac. Sci. Technol. 20, 316; Singhvi et al., Science (1994) 264, 696-698; Aplin and Hughes, Analyt. Biochem. (1981), 113, 144148; U.S. Pat. Nos. 5,324,591, 6,103,479, 4,729,949; U.S. Pat. Appl. No. US 99/04473; and PCT Appl. Nos. WO 03/035824; WO 99/45357].

In order to use standard equipment available in the art for handling multiwell microtiter plates (e.g., robotic plate handlers, robotic fluid dispensers, multipipettes, multifilters), a picowell-bearing component which bears a plurality of picowells is placed in at least one well of a standard multiwell microtiter plate.

A suitable distinct picowell-bearing component is a carrier including a plurality of picowells disposed on a surface, such as the carrier described in PCT patent application IL01/00992 or in unpublished copending PCT patent application IL04/00571 of the Applicant filed 27 Jun. 2004 or in unpublished copending PCT patent application IL2004/00061 of the Applicant filed 20 Jul. 2004. Picowell-bearing components are made of any suitable material, including reversibly deformable materials and irreversibly deformable materials. Suitable materials include but are not limited to gels, hydrogels, waxes, hydrocarbon waxes, crystalline waxes, paraffins, ceramics, elastomers, epoxies, glasses, glass-ceramics, metals, plastics, polycarbonates, polydimethylsiloxane, polyethylenterephtalate glycol, polymers, polymethyl methacrylate, polystyrene, polyurethane, polyvinyl chloride, rubber, silicon, silicon oxide and silicon rubber.

Preferably, the picowel-bearing component is composed of a gel, preferably a transparent gel, preferably a hydrogel. The advantage of using gels is that the diffusion of the secreted molecule is slowed-down, allowing identification of which cell secreted the molecule, thereby allowing kinetic evaluations.

Gels suitable for use in making a picowell-bearing component of a plate of the present invention include but are not limited to agar gels, agarose gels, gelatins, low melting temperature agarose gels, alginate gels, room-temperature Ca²⁺-induced alginate gels and polysaccharide gels. The gel may have a water content of greater than about 80% by weight, greater than about 92% by weight, greater than about 95% by weight, greater than about 97% by weight and even greater than about 98% by weight.

Preferably, the picowells in a given well (e.g., microwell) are juxtaposed, essentially meaning that the interwell area (i.e., between picowells) in the picowell bearing component is minimized to avoid cell adhesion outside of the picowell. Thus, preferably the inter-well area between two picowells is less than or equal to 0.35, 0.25, 0.15, 0.10 or even 0.06 of the sum of the areas of the two picowells. In certain embodiments of the present invention it is preferred that the inter picowell area be substantially zero, that is that the rims of picowells are substantially knife-edged.

Furthermore, to avoid cell adhesion or growth outside of the picowell bearing component, substantially the entire bottom surface of a microwell is covered by picowells

As mentioned, the dimensions of picowells of a multiwell plate of the present invention, depend on the type of cells used (i.e., prokaryotic vs. eukaryotic) and intended use thereof. Thus, picowells of the present invention are preferably less than about 200 microns, more preferably less than about 100 microns, even more preferably less than about 50 microns, yet more preferably less than about 25 microns or even less than about 10 microns.

The volume of the picowells of a multiwell plate of the present invention is typically less than about 1×10⁻¹¹ liter, less than about 1×10⁻¹² liter, less than about 1×10⁻¹³ liter, less than about 1×10⁻¹⁴ liter or even less than about 1×10⁻¹⁵ liter.

The area of the first cross section of such a picowell is typically less than about 40000 micron², less than about 10000 micron, less than about 2500 micron, less than about 625 micron² or even less than about 100 micron².

To avoid the heterogenic behavior of the cells of the present invention, picowells are configured (e.g., size, volume wise) to hold no more than a pair of living cells (i.e., sensor cell and secretor cell) at any one time.

The multiwell plate of this aspect of the present invention may be configured to delay proliferation of cells held therein, for example, by delaying adhesion of living cells thereto. For example, the inside of a picowell may include a material that delays adhesion of living cells thereto, that is the picowell is substantially fashioned from the adhesion-delaying material or the inside of the picowell is coated with the adhesion-delaying material (e.g., polydimethylsiloxane).

Preferably, wells (pico or microwell) of the multiwell plate of the present invention are composed of a material having an index of refraction similar to that of water. Preferably, the index of refraction of the bottom surfaces is less than about 1.4, less than about 1.38, less than about 1.36, less than about 1.35, less than about 1.34 or substantially equal to that of water.

The inner surface of the wells of the multiwell plate of the present invention can be coated with a layer of a material, such as, but are not limited to gels, hydrogels, polydimethylsiloxane, elastomers, polymerized para-xylylene molecules, polymerized derivatives of para-xylylene molecules, rubber and silicon rubber.

To prevent cell leakage from one well (e.g., picowell, microwell) to another, especially when suspension cultures are used or when no physical or chemical attachment of the cells to the wells is performed, the plate of the present invention may further include a gel cover covering the wells. Suitable gels are described hereinabove.

Multiwell fabrication may be performed using any one of several art known techniques, including, but not limited to, photolithography, stamping techniques, pressing, casting, molding, microetching, electrolytic deposition, chemical or physical vapor deposition employing masks or templates, electrochemical machining, laser machining or ablation, electron beam machining or ablation, and conventional machining. As will be appreciated by those skilled in the art, the technique used will depend on the composition and shape of the substrate, as well as on sample volume.

Device 10 also includes one or more sample ports 106, each being in fluid communication with locations 102 via channels 104. Sample ports 106 serve for feeding sample 105 (gas or liquid) through channels 104 and into locations 102.

Although channels 104 can feed sample 105 to both secretor cells 110 and sensor cells 108, according to one embodiment of the present invention, channels 104 are preferably arranged in or on surface 103 in a manner which enables delivery of sample 105 to secretor cells 110 and not sensor cells 108. This ensures that secretor cells 110 are exposed to the sample while sensor cells 110 are not. When sample 105 is in a gaseous state, its components (e.g., organic components) are preferably bound to an aqueous phase prior to the feeding of sample port 106.

Surface 103 may also be coated with a material 109 which may support or inhibit cell growth.

In order to generate an addressable array, each cell type (108 or 110) is allowed to settle in a distinct and known location of locations 102. Sensor cells 108 and secretor cells 110 are diluted to a desired concentration such that a predetermined number of cells are dispensed in each location. The cells are then allowed to settle on support (e.g., microwell, picowell plate) of the present invention. Sensor cells 108 and secretor cells 110 can be applied to surface 103 using any method known in the art, such as adsorption, entrapment, covalent binding, cross-linking or a combination thereof known in the art, although it will be appreciated that the specific method(s) utilized will depend on the nature and type of locations 102. Since the present invention relies on the activity of viable cells, gentle immobilization techniques such as entrapment and adsorption are preferably utilized.

Cells are placed on support 101 such that the secretor cells 110 and sensor cells 108 are in fluid communication therebetween. Thus, secretor cells 110 and sensor cells 108 may be placed in separate wells having a membrane or gel barrier which allows transition of the molecule from one well to another, preferably a one-way direction. This will allow sensor cells 108 to interact with the molecules secreted from secretor cells 110 while keeping secretor cells 110 isolated from substances present in the environment of sensor cells 108.

Alternatively, secretor wells 110 and sensor wells 108 may be placed in a single location, in this case, measures are preferably taken to ensure that sensor cells will not respond to the substance. Thus, sensor cells may be placed in the location once the substance is removed.

Defined arrangement of secretor wells 110 and sensor wells 108 on support 101 and known distances of the secretor cells from the sensor cells will allow identification of the substance and determining level thereof. For example, numerous sensor cels may be placed at varying distances from at least one sensor cell and their signals may be used to study relative diffusion of the molecules secreted from the secretor cells.

A gellable fluid, which is capable of slowing down transfer of the molecule from the secretor cells to the sensor cells may be used. The sample may be diluted in such a gellable fluid. The gellable fluid is chosen such that upon gelling, a transparent gel is formed. In a preferred embodiment, the gellable fluid is chosen so that upon gelling a hydrogel is formed.

Depending on the nature of the gellable fluid used, preferred methods of gelling the gellable fluid include of heating the gellable fluid, cooling the gellable fluid, irradiating the gellable fluid, illuminating the gellable fluid, contacting the gellable fluid with a gelling reagent and waiting a period of time for the gellable fluid to gel. Gellable fluids suitable for use in implementing the method of the present invention include but are not limited to agar gel solutions, agarose gel solutions, gelatin solutions, low melting temperature agarose gel solutions, alginate gel solutions, room-temperature Ca²⁺-induced alginate gel solutions and polysaccharide gel solutions. Depending on the embodiment, a gellable fluid has a water content of greater than about 80% by weight, greater than about 92% by weight, greater than about 95% by weight, greater than about 97% by weight and even greater than about 98% by weight. A preferred gellable fluid is an alginate solution where gelling the gellable fluid includes contacting the gellable fluid with a gelling reagent, such as a gelling reagent including Ca²⁺ ions. An additional preferred gellable fluid is a low melting temperature agarose solution and gelling the gellable fluid includes cooling the gellable fluid.

Sensor cells 108 and secretor cells 110 are maintained viable on support 101 using any growth medium which matches the nutritional needs of the cells used [see ATCC quality control methods for cell lines (2^{nd} ed.) American Type Culture Co. (Rockville, Md.)]. Increasing intracellular compatible solute concentration [e.g., by active import from the intracellular environment (e.g., uploading with non-metabolizable sugars) or by inducing autosynthesis (e.g., genetic engineering, growth in high salinity medium)] may be preferred since it is well established that accumulation of compatible solutes, may provide enhanced resistance to freezing and drying which may be used to maintain cells during storage. Examples of compatible solutes include, but are not limited to, glycine, proline, hydroxyectoine and trehalose.

As mentioned hereinabove, cells may be dispensed on support 101 to provide an array of at least 2 cells (sensor and secretor cells). However, arrays having high cell density may be preferred since signals generated from such cells increase in proportion to the number of cells utilized.

Typically, the cells are retained in close proximity to the detector by using membranes such as a dialysis membrane. In general, the outer membrane is chemically and mechanically stable, with a thickness of 10-15 μm and a pore size of 0.1-1 μm. Preferably used are pore trace membranes made of polycarbonate or polyphthalate. Other immobilization methods are described in U.S. Pat, No. 6,692,696.

When sample 105 is in a fluid state, locations 102 are preferably configured as reaction chambers 109. Reaction chambers 109 are preferably addressable so as to allow addressable monitoring thereof, as further detailed hereinunder.

Fluid channels 104 are preferably microfluidic channels. Techniques of forming microfluidic channels in a substrate are known in the art and several protocols have been proposed for such formations [to this end see, e.g., Heusckel, M. O. et al, “Buried microchannels in photopolymer for delivering of solutions to neurons in a network”, Sensors and Actuators B 48:356-361, 1998]. For example, the micro channels may be formed by micro-lithography. Transport of sample 105 from sample port 106 through channels 104 and into or onto reaction chambers 109 can be effected using a variety of methods which are known in the art.

There are many techniques for actuating fluid transport through microchannels. One example of a mechanism suitable for transporting sample 105 to reaction chambers 109 is illustrated in FIG. 32c (indicated by numeral 602). Mechanism 602 can be a pump or an injector capable of pumping or injecting a sample fluid through channels 104 and into or onto reaction chambers 109. Any pumps or injector can be used, such as those disclosed in U.S. Pat. Nos. 6,033,191 and 6,460,974. Mechanism 602 can be placed on or in device 10 or not, depending on considerations such as costs, size of support 101 and the like. In any case, mechanism 602 is in fluid communication with sample port 106 and reaction chambers 109 to enable sample 105 delivery to reaction chambers 109.

Mechanism 602 preferably enables sample 105 delivery by applying a negative pressure to channels 104 reaction chambers 109, thereby delivering sample 105 from sample port 106 to reaction chambers 109.

As used herein “negative pressure” refers to a pressure value, which is smaller than a pressure value in a reference volume. For example, with respect to sample port 106, “negative pressure” refers to a pressure value which is smaller than the pressure value in sample port 106. The terms “negative pressure” and “under-pressure” are interchangeably used herein.

As is mentioned hereinabove, sensor cells 108 of the present invention respond to presence of a particular molecule (secreted from secretor cells 110) with a detectable signal. Thus, to enable detection of such cell generated signals, device 10 of the present invention forms a part of a system capable of detecting presence, absence or level of a substance by qualifying and optionally quantifying optical signals generated by sensor cells 108 of device 10.

Thus, the present invention provides populations of cells, which can be utilized for substance detection and qualification of an effect thereof on live cells (i.e., secretor cells).

It should be noted that since the present invention enables detection of any secreted molecule, the paired cell populations of the present invention can also be used to identify specific cells of a population of cells, based upon the capability of the specific cells to secret the molecule. Such an approach can be utilized to screen a population of transformed cells for a subpopulation, which expresses a recombinant protein of interest, or to screen a population of cells for a subpopulation, which expresses a specific variant of the recombinant protein of interest.

This can be effected by exposing sensor cells to a plurality of cells potentially capable of secreting the molecule of interest and identifying the cells expressing the molecule of interest.

It will be appreciated that the above methodology can also be implemented for cell classification. For example, it is well established that diseased cells, such as cancer cells are featured by a different protein profile than normal cells. For example, gastric cancer is one of the most common human cancers and is the second most frequent cause of cancer-related death in the world. Serial analysis of gene expression (SAGE) showed that regenerating gene type IV (REGIV) is upregulated in scirrhous-type gastric cancer. RegIV is secreted by cancer cells and inhibits apoptosis, rendering RegIV an important biomarker for gastric cancer [Yasui (2004) Cancer Sci. 95(5):385-92]. Thus, by screening cells for secretion of RegIV and analyzing apoptosis in sensor cells one can classify cells as gastric cancer cells. This method has obvious diagnostic and therapeutic implications.

Cells of the present invention may be packed in a kit. For longterm storage, cells of the present invention may be dried. Drying formulations may include bulking agents, cryoprotectants, lyoprotectants, sugars and the like, which are preferably present both inside and outside of the cells.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 14, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. p317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Real-Time Quantification of ROS and NOS Levels According to the Teachings of the Present Invention

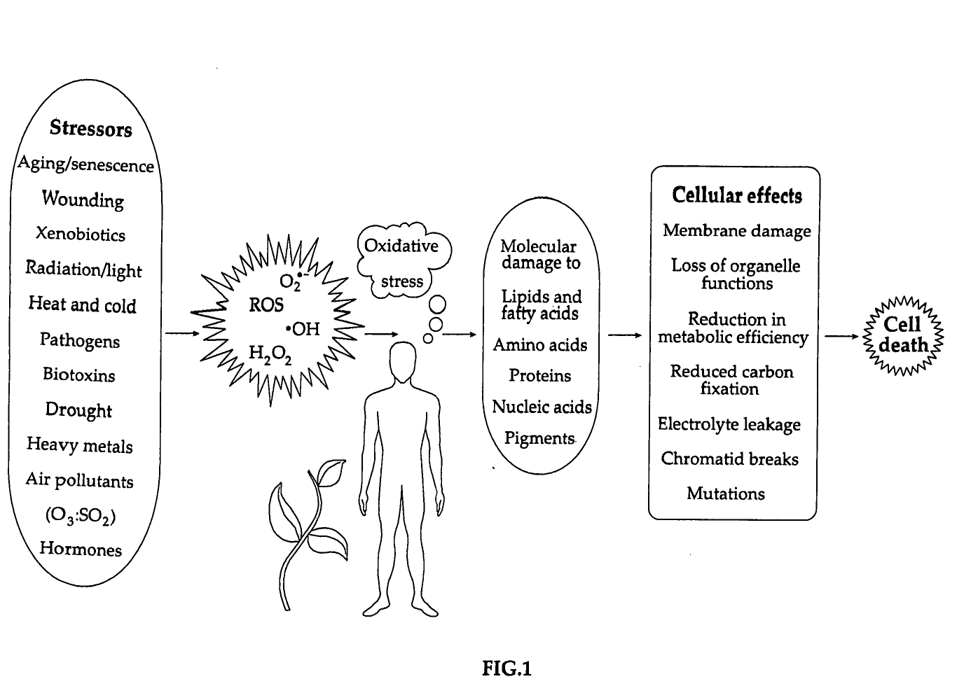

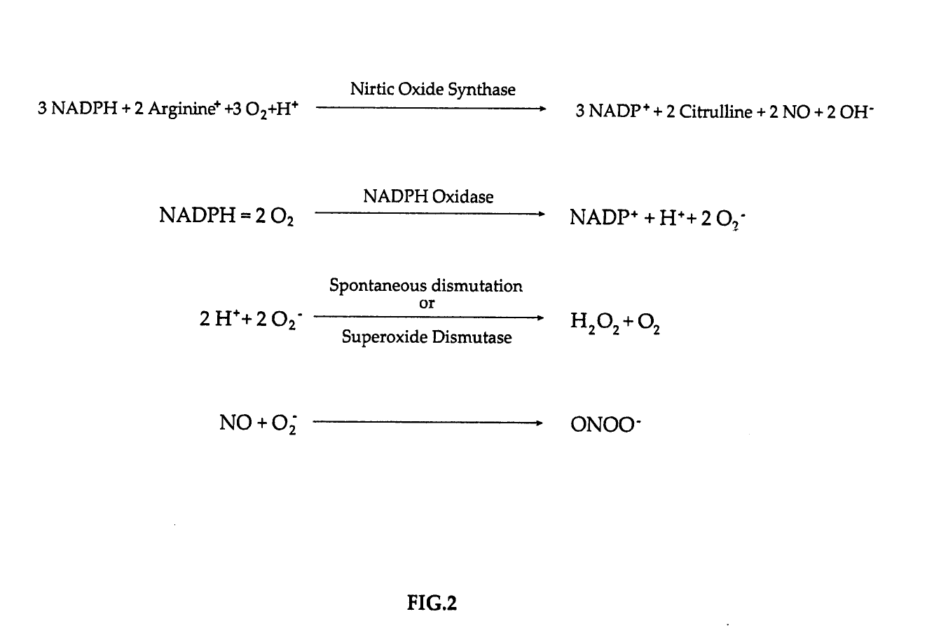

Many free radical reactions are highly damaging to cellular components, i.e., they crosslink proteins, mutagenize DNA, and peroxidize lipids (see FIGS. 1 and 2). Once formed, Reactive Oxygen Species (ROS) and Reactive Nitrogen species Species (RNS) can interact to produce other free radicals and nonradical oxidants such as singlet oxygen (¹O₂) and peroxides. Degradation of some of the products of free radical reactions can also generate potentially damaging chemical species. For example, malondialdehyde is a reaction product of peroxidized lipids that reacts with virtually any amine-containing molecule. Oxygen free radicals also cause oxidative modification of proteins [Stadtman, E. R. (1992) Science 257:1220), see U.S. Pat. Nos. 6,589,948].

In-vitro detection of gaseous Reactive Oxygen Species (ROS) and Reactive Nitrogen species Species (RNS) compounds secreted by a population of heterogeneous cells has been difficult in the past. One difficulty in accurate detection/measurement is that the half life of ROS and NOS is short. This requires monitoring them as close as possible to the cells that secrete them before they have a chance to react with other molecules and change their molecular nature making detection/measurement infeasible.

Cell populations show significant heterogeneity both in their baseline levels of ROS and NOS production and in their rates of response to applied stimuli. Additionally, different cells within the population may exhibit different kinetics of ROS generation. All of these factors make it difficult to learn about any one particular type of cell within the population as any measured gas/small molecules could have been secreted from any cell at any time prior to the measurement.

Additionally, NO is a small molecule which diffuses rapidly across cell membranes and, depending on conditions, is able to diffuse across distances of several hundred microns, so when NO is measured it is difficult to ascertain where the NO molecule originated from.

Furthermore the balance between oxidative and nitrosative stress is controlled by a ratio of production of ROS and NOS. It is therefore significant to be able to simultaneously measure ROS and NOS rates of formation.

### EXAMPLE 1

### Qualification of NOS Indicators

Materials and Experimental Procedures

Materials—Diethylene triamine NONOate (DETN/NO) was purchased from Alexis Biochemical (Alexis Corporation, UK). 4,5-diaminofluorescein diacetate (DAF-2DA) was purchased from Calbiochem (La Jolla, Calif.).

Cells—U937 pro-monocyte cells were obtained from DSMZ-German Collection of Microorganisms and Cell Cultures; Department of Human and Animal Cell Cultures Braunschweig, Germany. U937 cells were maintained in RPMI-1640 medium supplemented with 10% heat-inactivated fetal calf serum, 100 U/mL penicillin, 100 μg/mL streptomycin, 2% glutamine, 2% sodium pyruvate and 2% HEPES (complete medium, all materials were obtained from Biological Industries, Kibbutz Beit Haemek, Israel). Cells were maintained in completely humidified air with 5% CO2 at 37° C. [Kinscherf, R, Claus R, Wagner M, Gehrke C, Kamencic H, Hou D, Nauen O, Schmiedt W, Kovacs G, Pill J, Metz J and Deigner HP (1998) FASEB J. 12(6), 461-467].

Cell loading—For probe loading, 100 μl of cells (1.5-2×10⁶ cells/mL of PBS or serum free media without phenol red) were incubated in the presence of 10 μM DAF-2DA for 15 min at 37° C. and 5% CO2, and then washed in PBS.

Fluorescent microscopy and imaging system—Olympus motorized upright epi-fluorescence BX51 microscope, equipped with motorized polarization filters for fluorescence polarization measurements was used. Cells were illuminated by a Mercury light source. The emitted fluorescence was imaged by CoolSNAP HQ monochrome CCD camera, or DVC-1312 (DVC Company, West Austin, Tex., USA) color camera. Digital image analysis of cellular fluorescence was performed by Image Pro plus software (Media Cybernetics. Inc. Silver Spring, Md.,USA).

Results

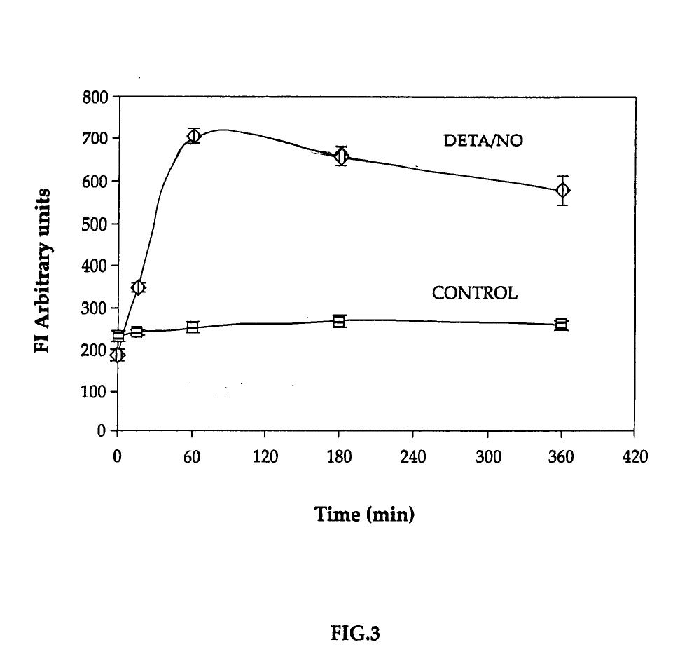

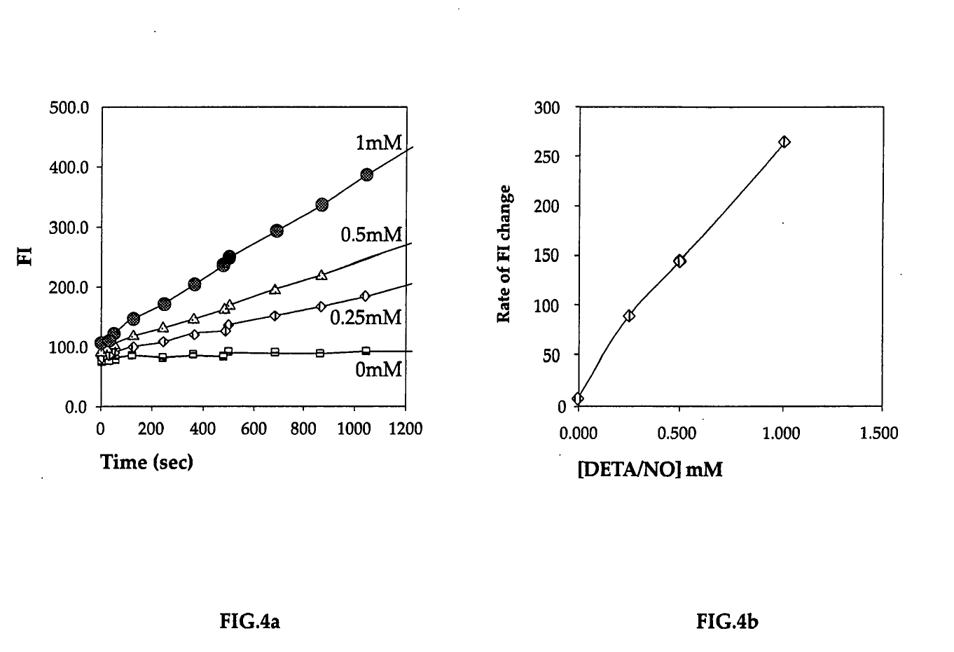

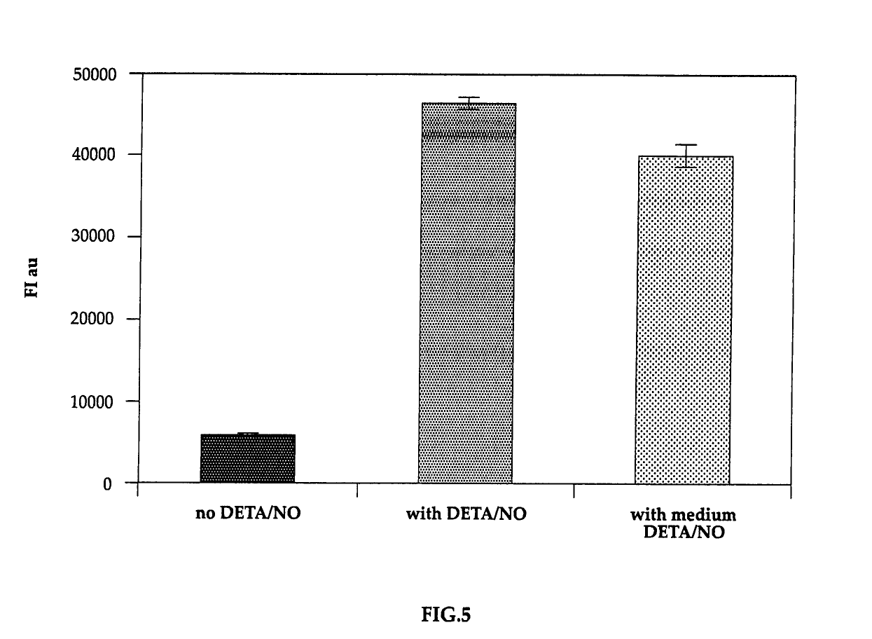

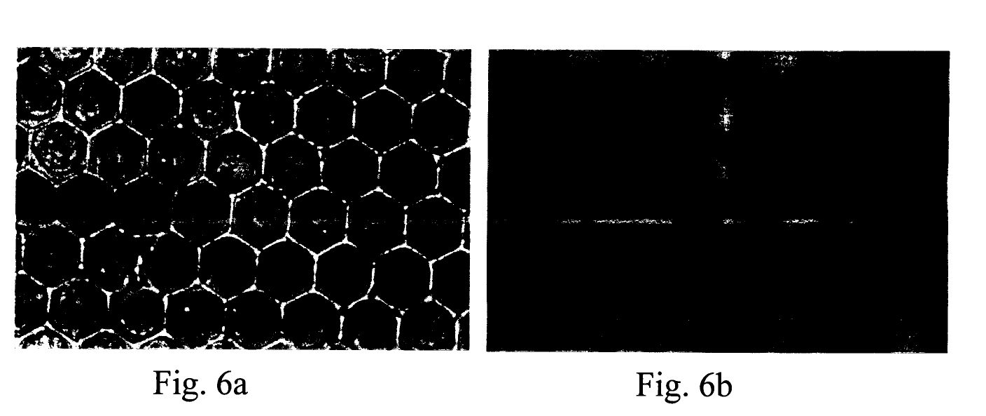

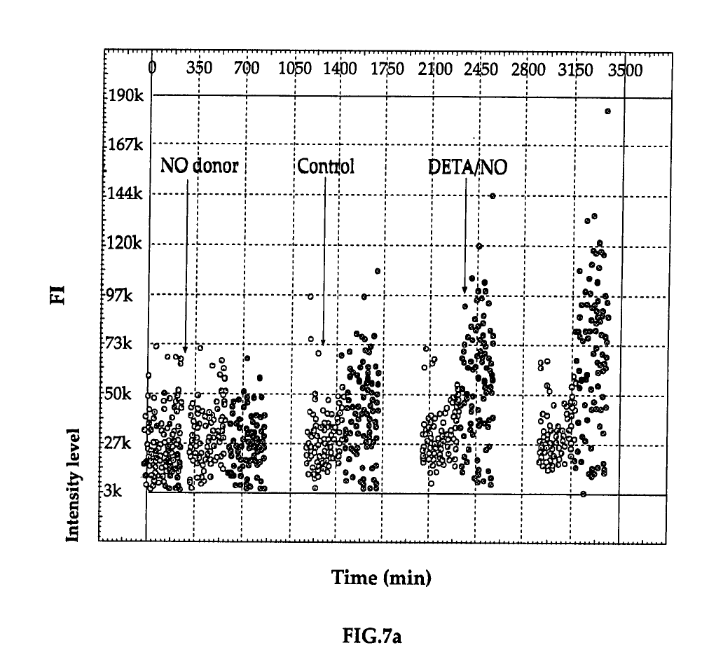

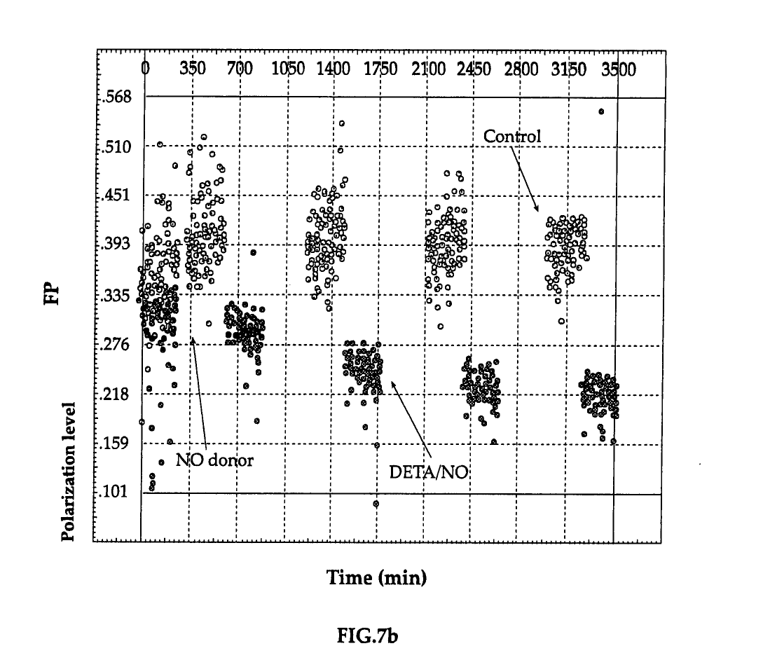

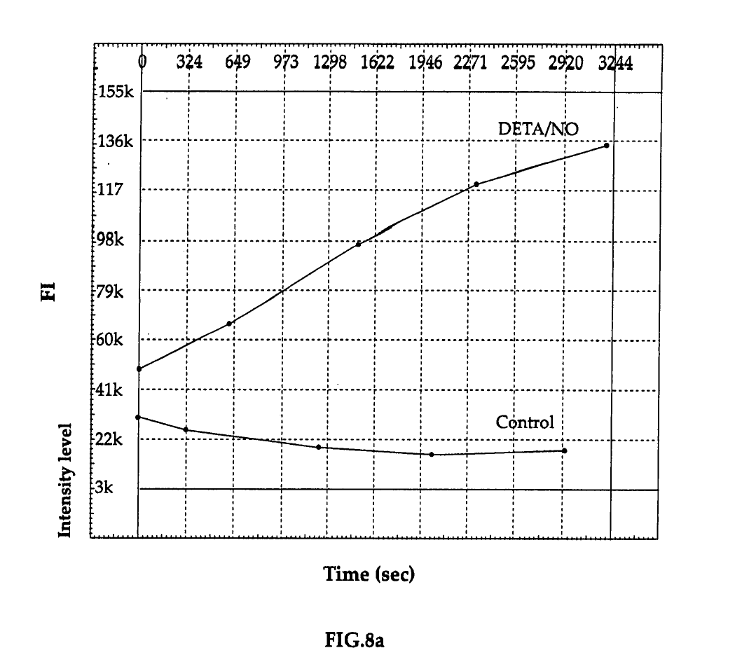

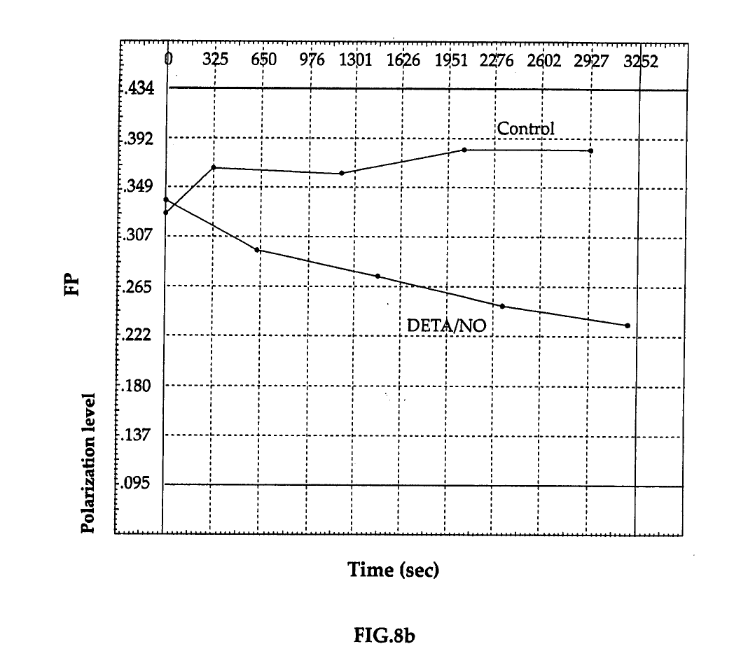

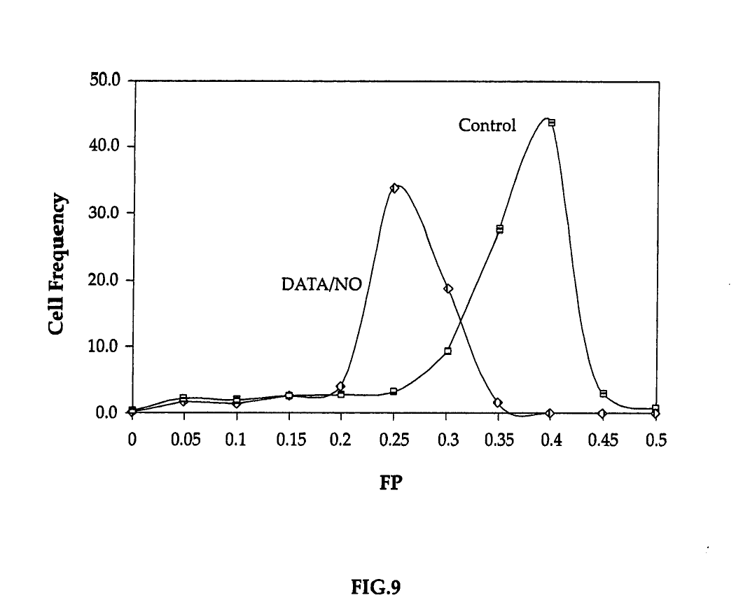

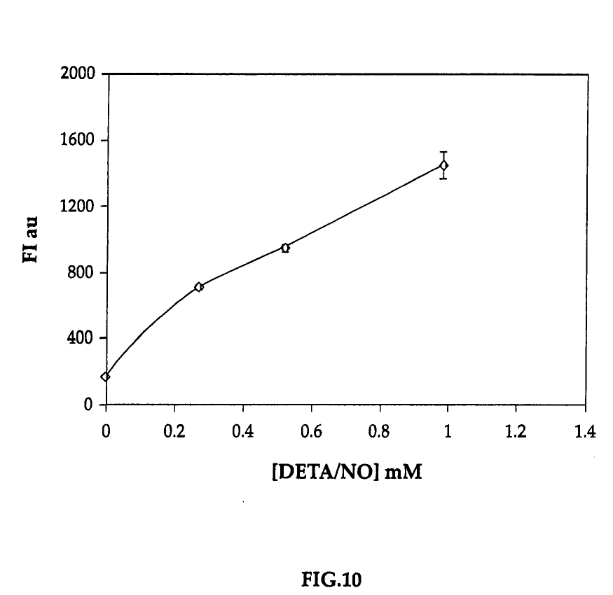

Quantitative measurements of intracellular NO concentration in individual U937 cells were effected in response to incubation in the presence of the Diethylene triamine NONOate (DETN/NO) donor (0.1-1 mM). The loading of cells with probe was measured in bulk at the individual cell level. Probe concentration loading time and cell washing were determined. The kinetic of NO generation was measured in real time by sequentially monitoring the same individual cells. Photo-toxicity induced by repeated excitations was checked and subtracted. Time response of NO generation in the presence or absence of DETA/NO donor (0.5 mM) in U937 cells labeled with DAF-2DA (10 μM, 15 min) is shown in FIG. 3. Dose response of NO generation by the DETA/NO donor is shown in FIGS. 4a-b. FIG. 5 shows NO generation in DA-2FDA-labeled U937 cells in the absence (no DETA/NO) or presence of donor (with DETA/NO) or in the presence of U937 conditioned-medium pre-incubated with DETA/NO for 1 h (with medium DETA/NO). These results demonstrate the ability of U937 cells to accumulate NO in response to the DETA/NO donor. These results were further substantiated by fluorescent microscopy of individual cells stained with DAF-2DA and incubated for 1 hour in the presence of DETA/NO (FIGS. 6a-b). Quantitative measurements of intracellular NO levels in individual living cells labeled with DAF-2DA and treated with DETA/NO are shown in FIGS. 7a-b and 8a-b. FIG. 9 shows FP distribution patterns of individual U937 cells labeled with DAF-2DA in the presence or absence of an NO donor. The change in Fl following 1 hour incubation with different concentrations of DETA/NO is shown in FIG. 10.

Altogether these results qualify the DAF-2DA probe and U937 cells as a good system for sensitively measuring reactive nitrogen species (RNS).

### EXAMPLE 2

### Qualification of ROS Indicators

Materials and Experimental Procedures

Materials—Lysophosphatidylcholine (LPC—L-A LPC type V containing primarily palmitic, stearic and oleic acids), hydrogen peroxide (H2O2), superoxide dismutase (SOD), methotrexate (MTX) were obtained from Sigma-Aldrich (St.Louis, Mo., USA).Dihydrorhodamine 123 (DHR123), 2,7-dichlorofluorescein diacetate (DCFDA), dihydroethidium (DHE), were purchased from Calbiochem (La Jolla, Calif.).

Cells—U937 were described in Example 1 above. THP-1 cells were maintained in RPMI-1640 medium supplemented with 10% heat-inactivated fetal calf serum, 100 U/mL penicillin, 100 μg/mL streptomycin, 2% glutamine (Biological Industries, Kibbutz Beit Haemek, Israel). Cells were maintained in completely humidified air with 5% CO₂ at 37° C.

Measurement of intracellular ROS in live cells—The measurement of intracellular reactive oxygen species (ROS) levels was performed using the DHR123 (1-10 μM), DCFDA(1 μM) or DHE (2 μM) probes.

For probe loading, 100 μl of cells at a concentration of 1.52×10⁶ cells/mL in PBS or in serum free media without phenol red were incubated in the presence of the various probes for 15 min at 37° C. and 5% CO₂, and then washed twice in PBS and thereafter exposed to either LPC (10-20 μM), MTX (50 nM) or H₂O₂ (10-500 μM).

Results

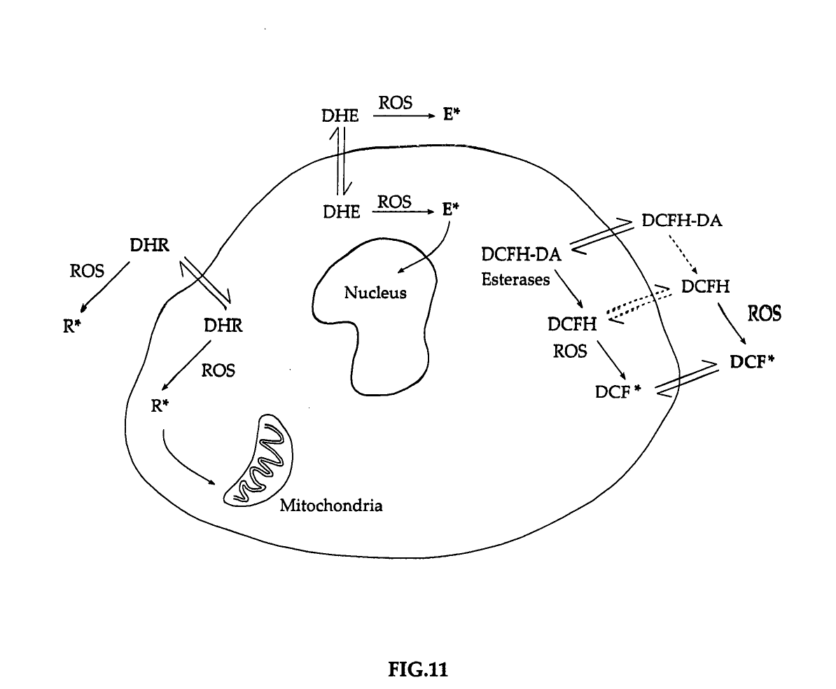

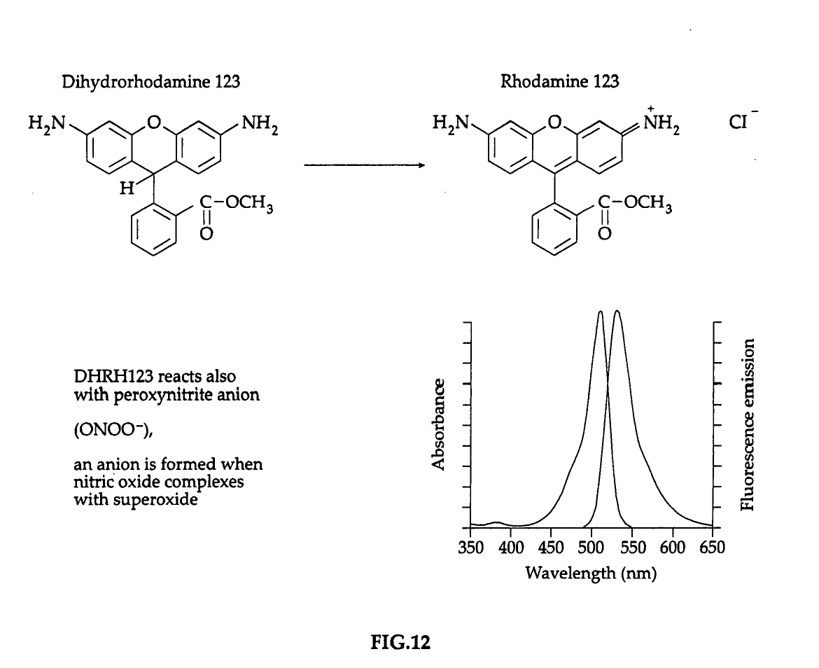

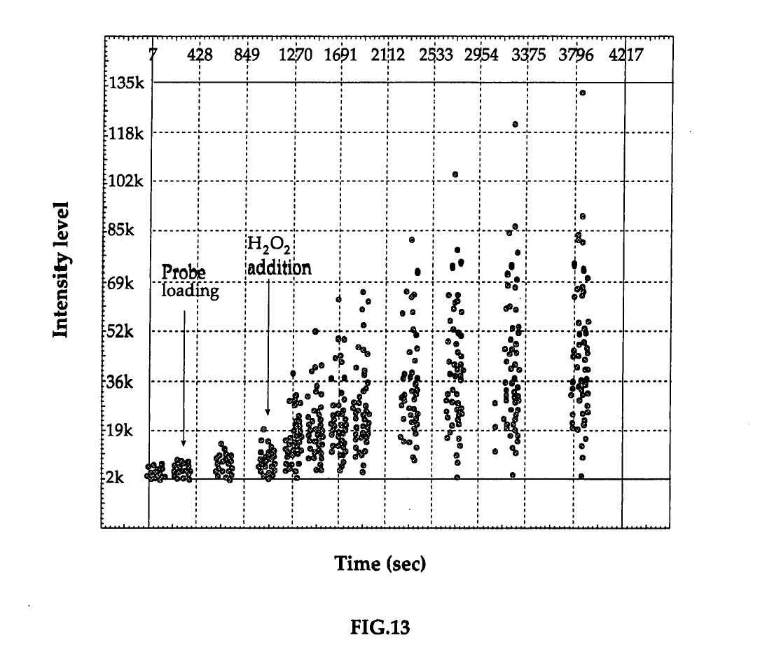

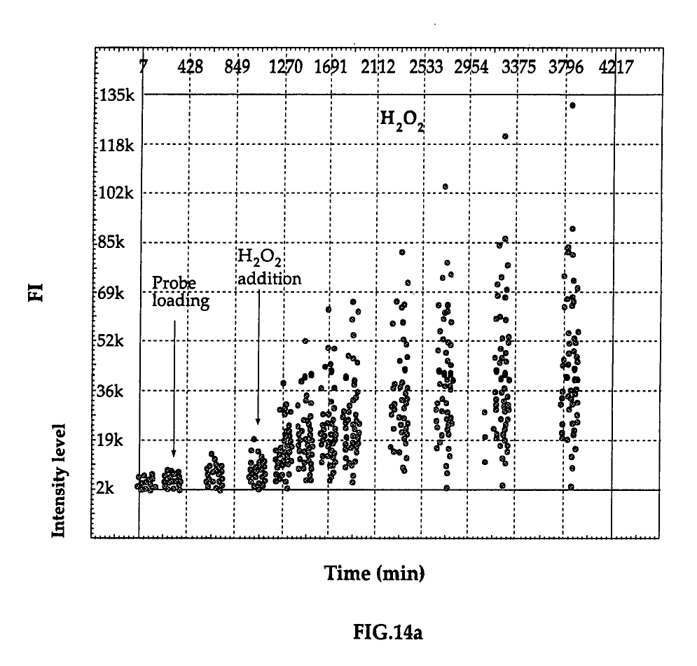

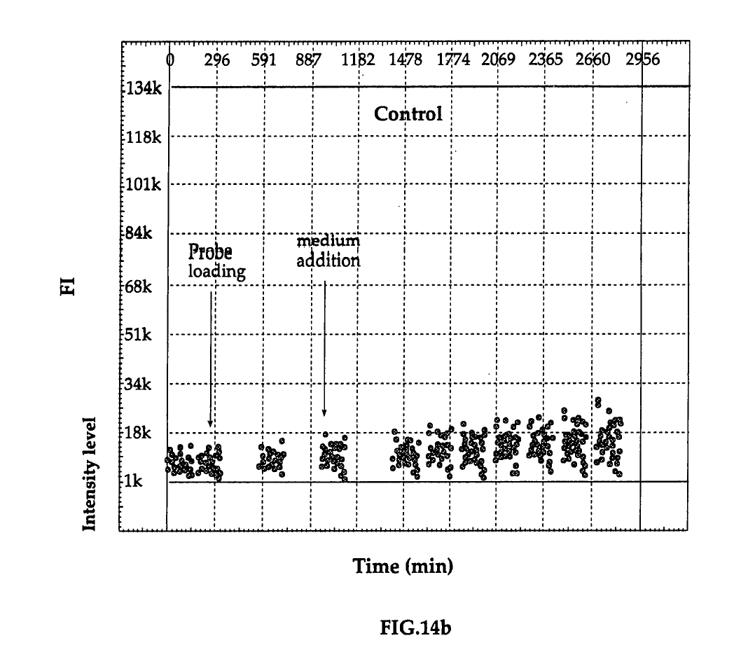

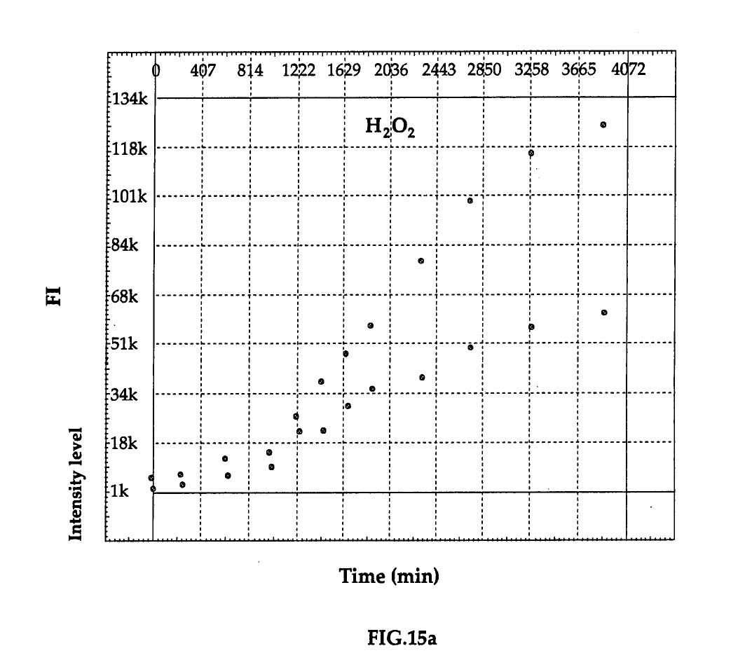

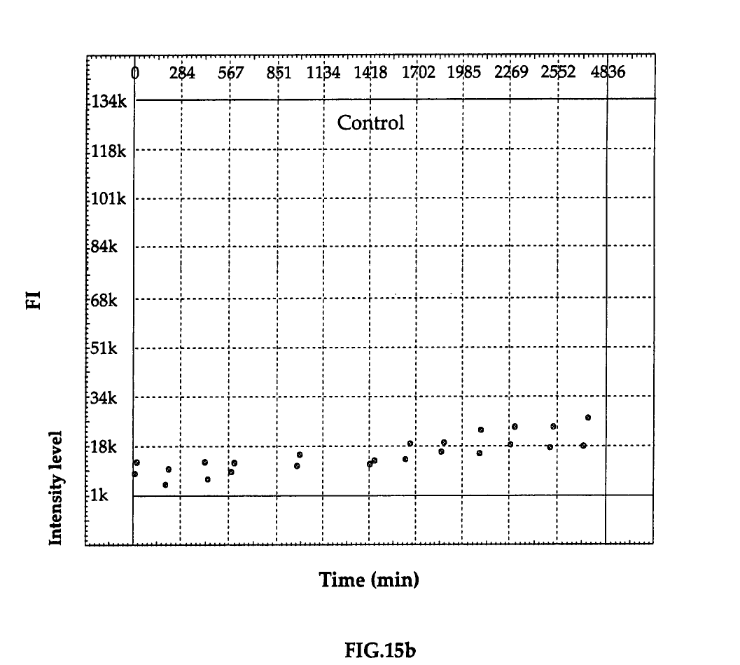

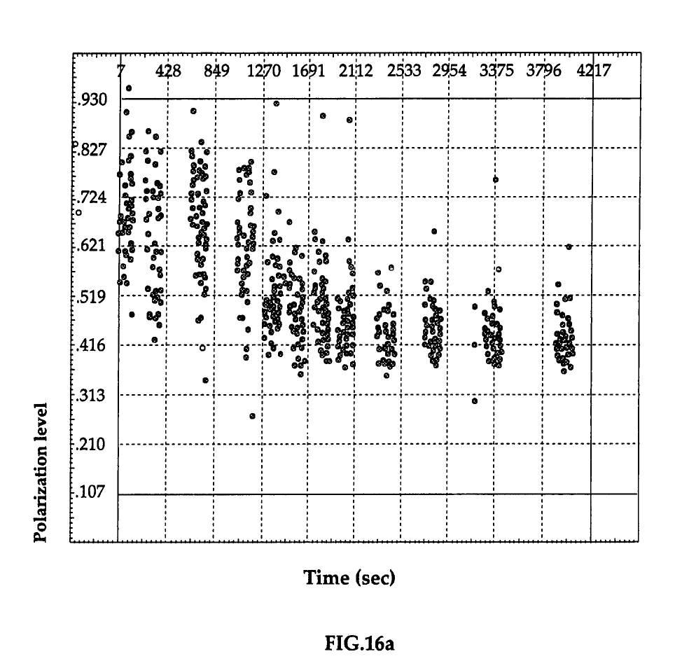

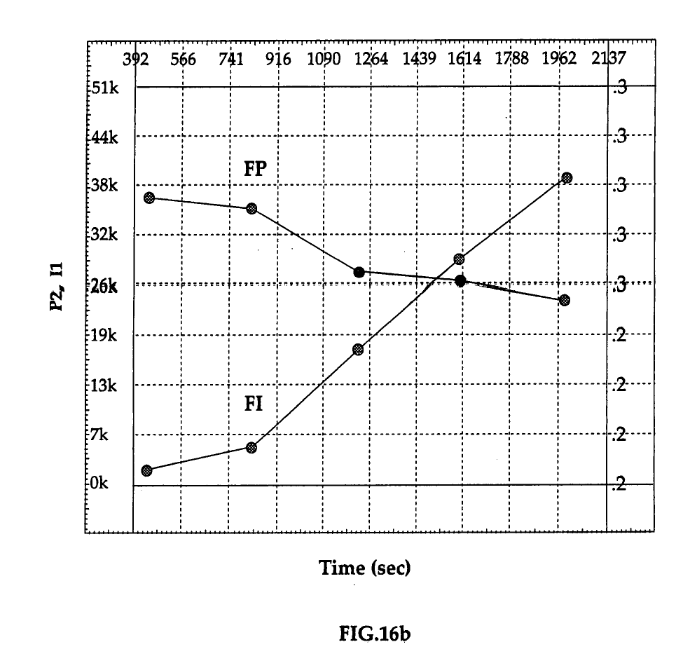

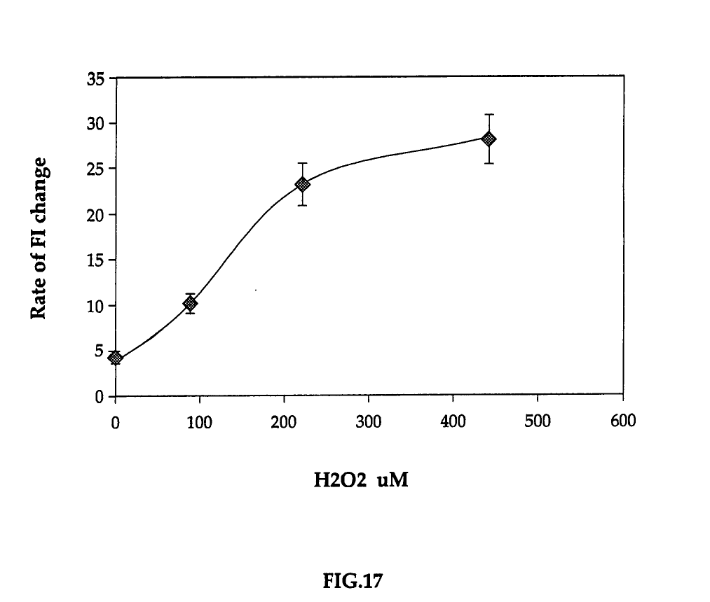

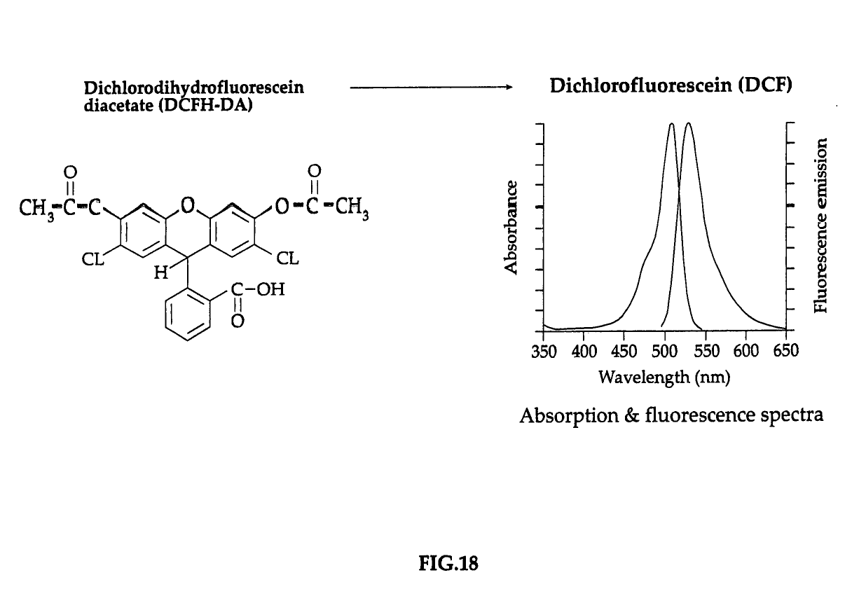

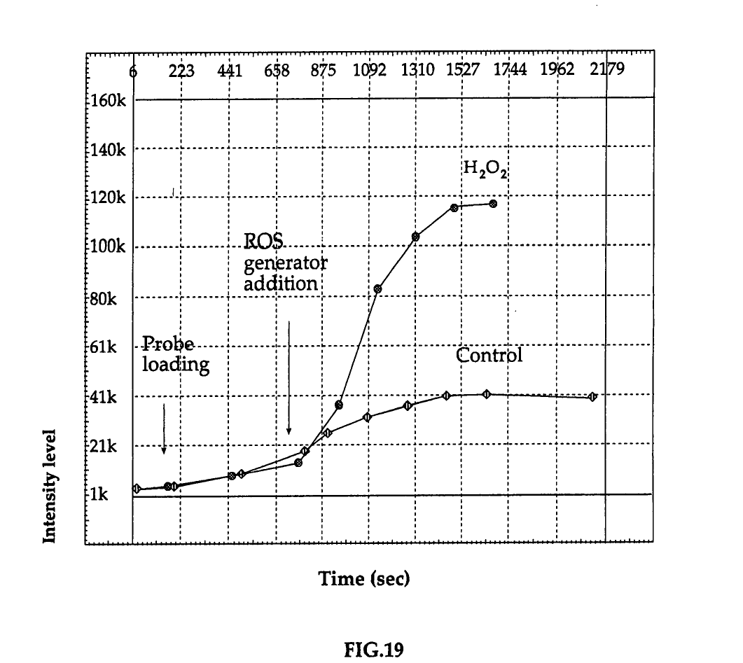

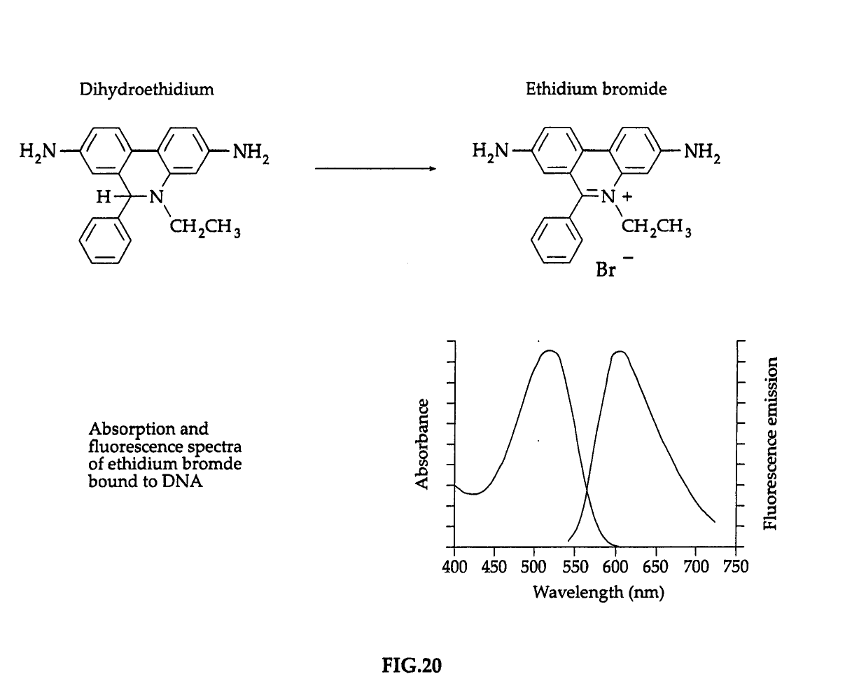

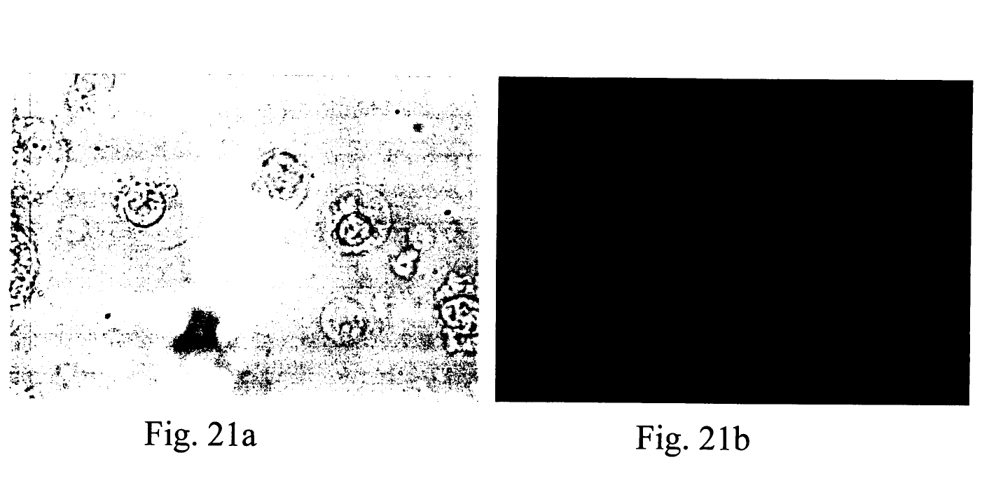

The pro-monocyte U937 cell line was used as cell culture model. Hydrogen peroxide was used as a source of ROS. Probe loading of cells was measured in bulk and at individual cell level. Probe concentration, loading time and cell washing were determined for the different ROS probes. The kinetics of ROS generation was measured in real time by monitoring sequentially the same individual cells. Photo toxicity induced by repeated excitations was checked. The cellular distribution of three ROS probes and their oxidized fluorescent species is shown in FIG. 11. FIGS. 12-17, 18-19 and 20-21a-b show the ability of dihydrorhodamine 123 (DHR), dichlorodihydrofluorescein diacetate (DCFH-DA) and dihydroethidium (DHE), respectively, to detect ROS formation in U937 cells treated with hydrogen peroxide. These results suggest that the above-described probes, which exhibited divers spectroscopic characteristics, can be used to detect ROS levels at different subcellular locations at different rates of formation.

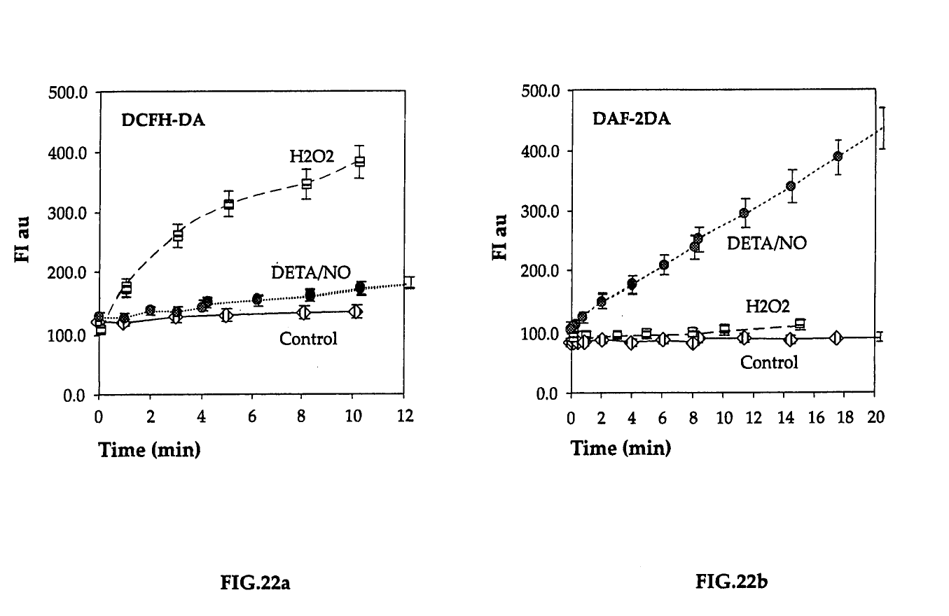

The specificity of DCFH-DA and DAF-2DA towards ROS and NOS respectively, was tested. As is shown in FIGS. 22a-b, cells labeled with DCFH-DA produced a strong and quantifiable signal in response to hydrogen peroxide while only a minor response was noted in the presence of the NO donor (see FIG. 22a-b). The opposite was shown in cells labeled with DAF-2DA, showing a strong signal in the presence of the NO donor while a background signal was shown in the presence of hydrogen peroxide (FIG. 22b).

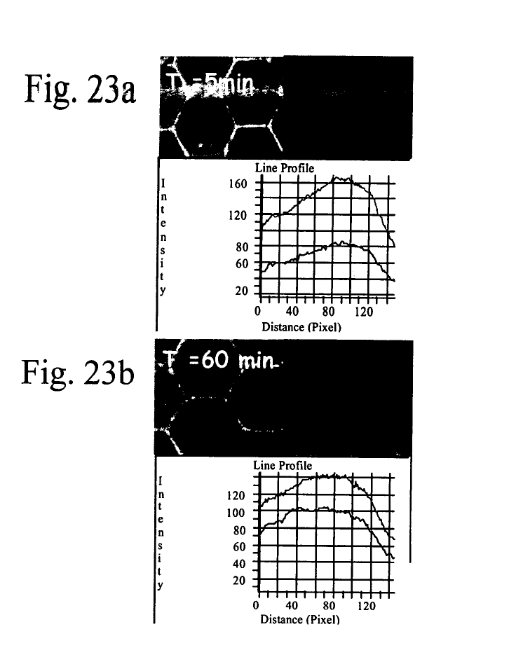

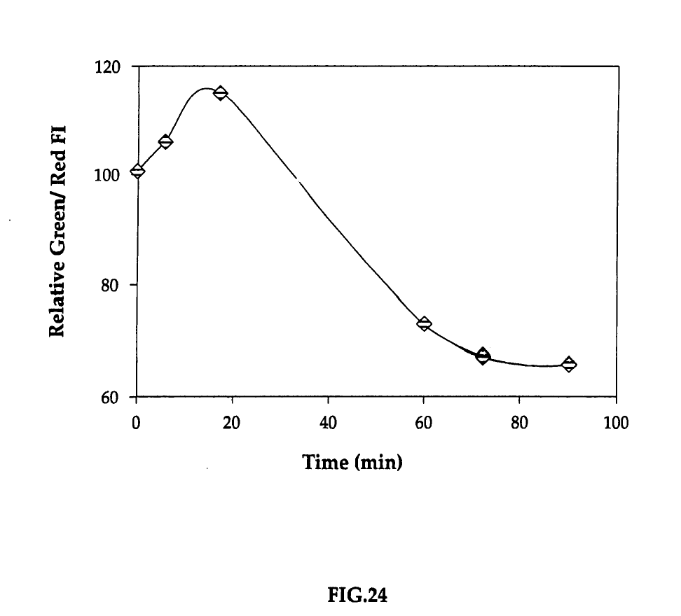

ROS generation was analyzed at the subcellular level. Individual THP1 cells were labeled with DHR123 and DHE. Temporal onset and spatial distribution of ROS in different intracellular locations were measured utilizing two probes in the same individual cells. Dual labeling with DHR123 (probe for mitochondrial ROS, green FI) and DHE (nuclear ROS, red FI) revealed that the onset of mitochondrial ROS generation exceeded that of the nucleus, since the Fl ratio (red FI/green FI) measurements first increased following the stimulus and decreased thereafter (FIGS. 23a-b and 24).

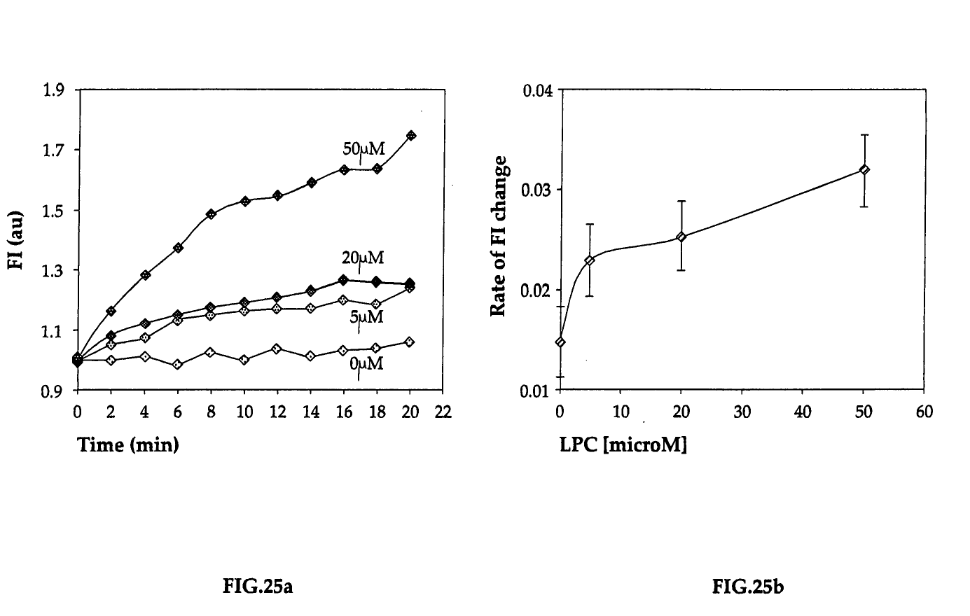

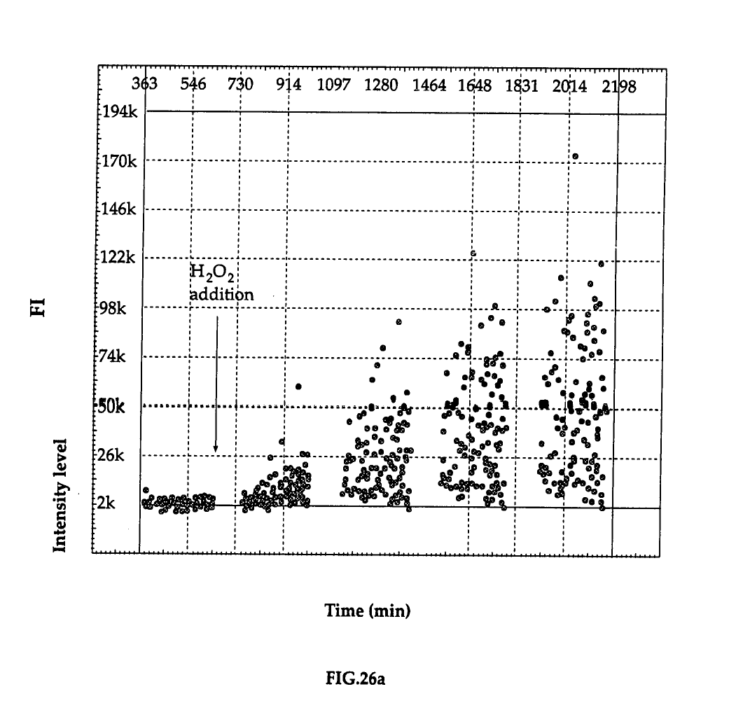

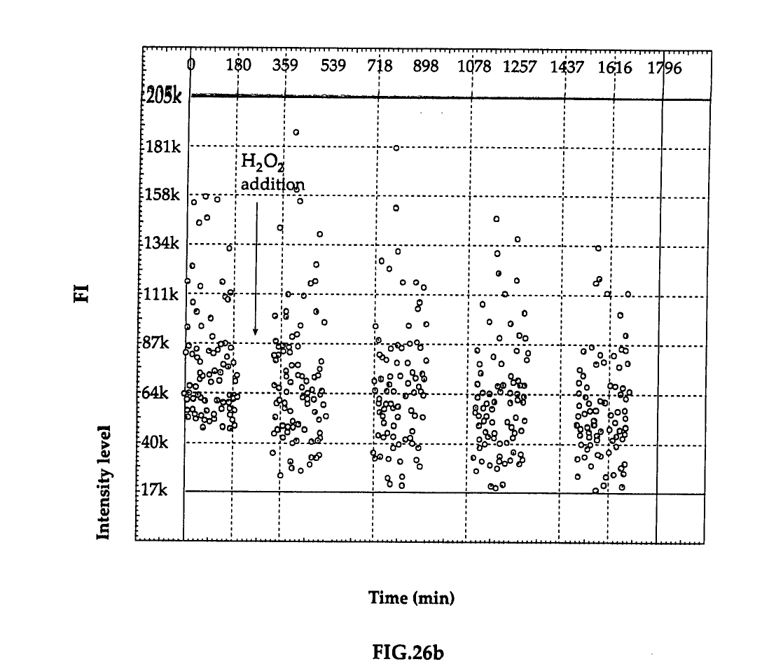

U937 cells labeled with DHR123 were incubated with lysophosphatidylecholine (LPC). Time and dose dependency of ROS production was then measured. Results are shown in FIGS. 25a-b.

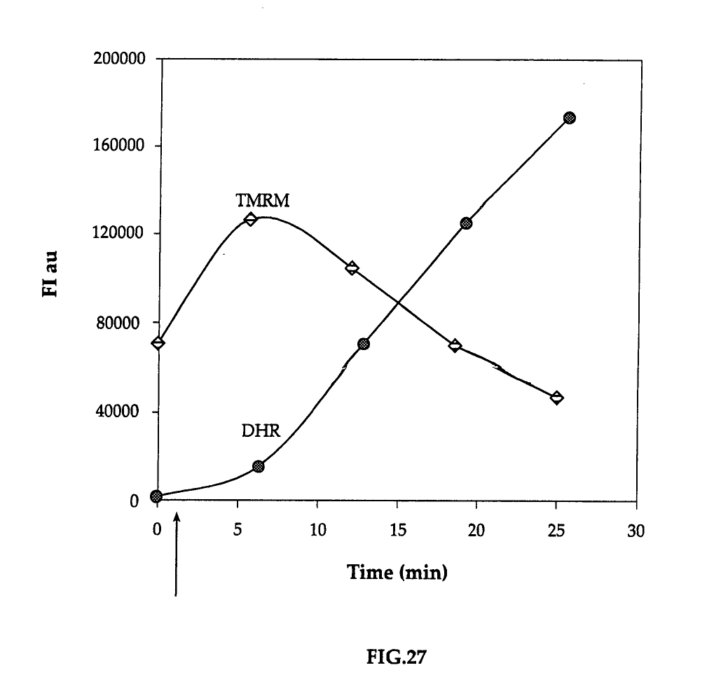

Disruption of mitochondrial membrane potential is one of the earliest intracellular events that occur upon apoptosis induction and may be accompanied by generation of free radicals [Waterhouse N.J., Goldstein J C, von Ahsen O, Schuler M, Newmeyer D D, Green D R. (2001), J. Cell Biol.;153(2):319-28]. As such DHR123 and Tetramethylrhodamine-methyl-ester (TMRM) were both used as detectors of ROS levels and mitochondrial membrane potential, respectively, in response to hydrogen peroxide stimulus (see FIGS. 26a-b). Temporal relationship between kinetic of ROS generation (measured by DHR) and the onset of changes in mitochondrial membrane potential (TMRM) in an individual live cell is shown in FIG. 27. Hyperpolarization of mitochondrial membrane potential preceded the increase in ROS level.

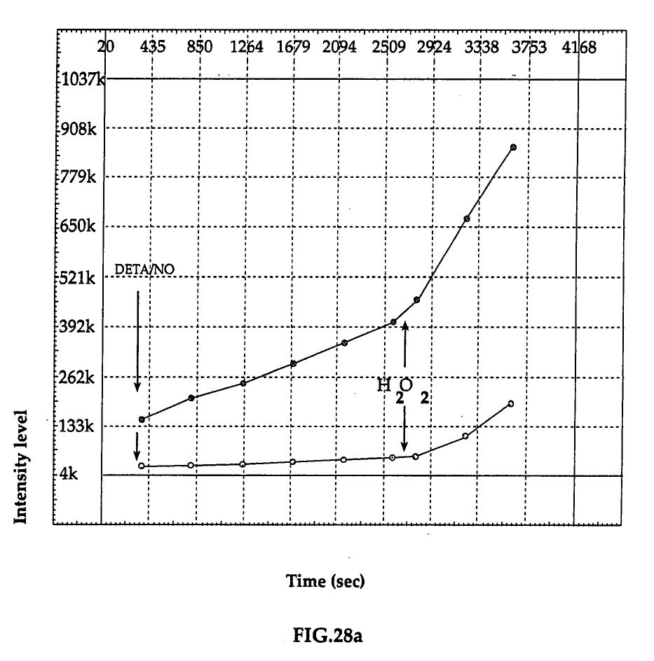

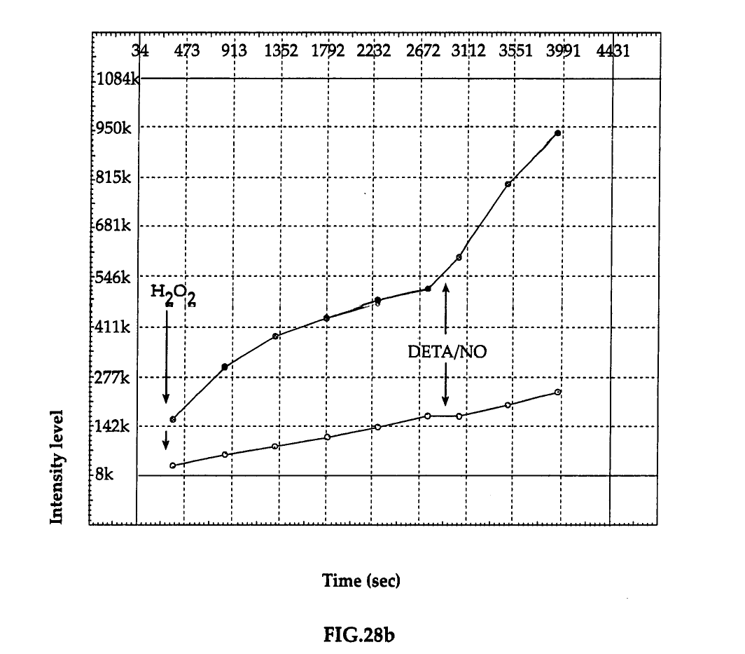

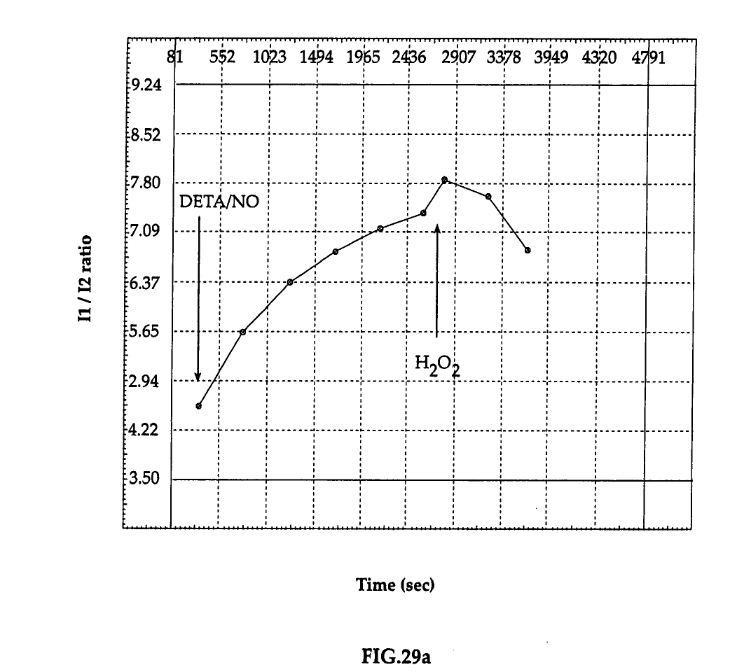

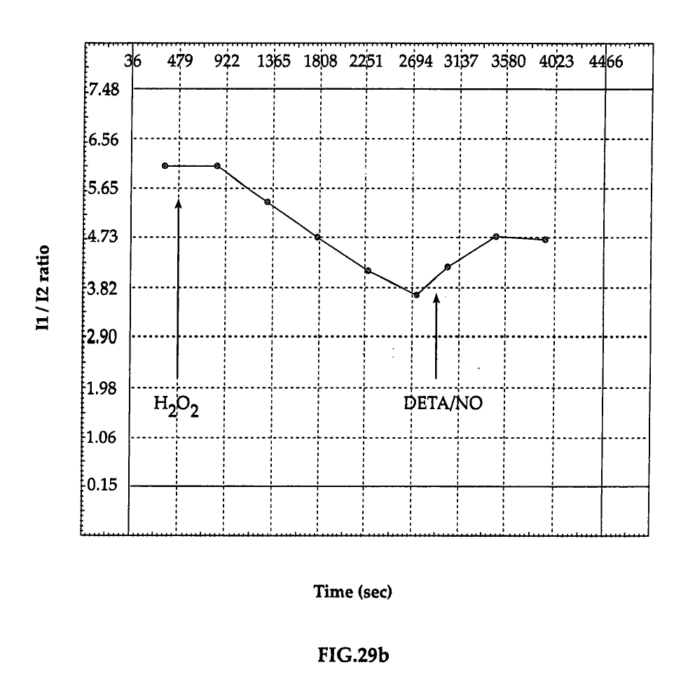

The ability of cells labeled with ROS and NOS probes to provide simultaneous measurement on ROS and NOS levels in response to respective hydrogen peroxide and NO donor stimuli, was measured. Results of FI and FIR measurements are shown in FIGS. 28a-b and 29a-b respectively. Addition of H₂O₂ following DETA/NO exposure is shown in FIG. 28a or vice versa, by the introduction of DETA/NO to H₂O₂ treated cells which is shown in FIG. 28b. An increase in FIR occurred when the rate of NO production exceeded the rate of ROS formation and decreased as the rate of ROS formation exceeded that of NO (FIGS. 29a-b).

### EXAMPLE 3

### Measuring ROS/INOS Production by Secretory Cells Using a Sensory Cell System

100 μl of U937 cells (1.5-2×10⁶ cells/ml in serum free media without phenol red) were incubated in the presence of DHR123 (2 μM) for 15 min at 37° C. and 5% CO₂, and then washed 3 times in PBS.

Activator cells (ROS secreting cells)-100 μl of unstained U937 cells (1.5-2×10⁶ cells/ml in serum free media without phenol red) were incubated in the presence of hydrogen peroxide (50 M) for 15 min at 37° C. and 5% CO₂, and then washed 3 times in PBS. It is well established that exogenous H₂O₂ elicits high intracellular ROS concentrations in U937 monocytes [Zurgil N, Solodeev I, Gilburd B, Shafran Y, Afrimzon E, Avtalion R, Shoenfeld Y, Deutsch M. Cell Biochem Biophys. 2004;40(2):97-113].

For monitoring ROS levels in sensor cells upon exposure to activated ROS secreting cells, stained sensor cells were loaded on the picowell device and a first (control) measurement was taken. Then, ROS secreting cells were loaded on the same pico-well device, and the kinetics of ROS generation was measured in real time by monitoring sequentially the same individual cells.

Results

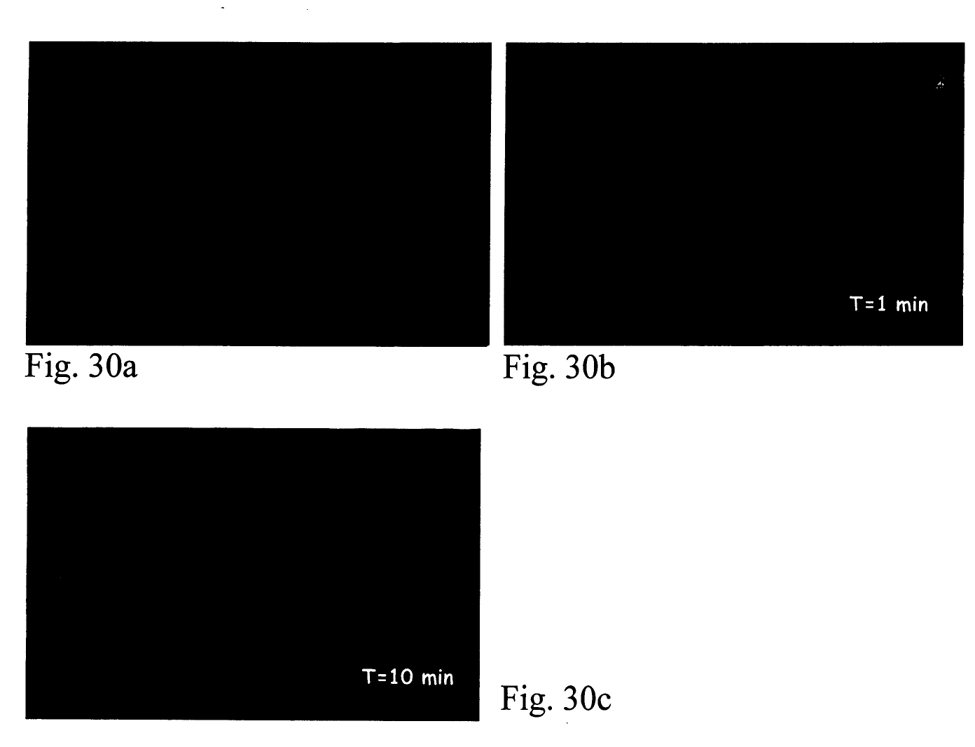

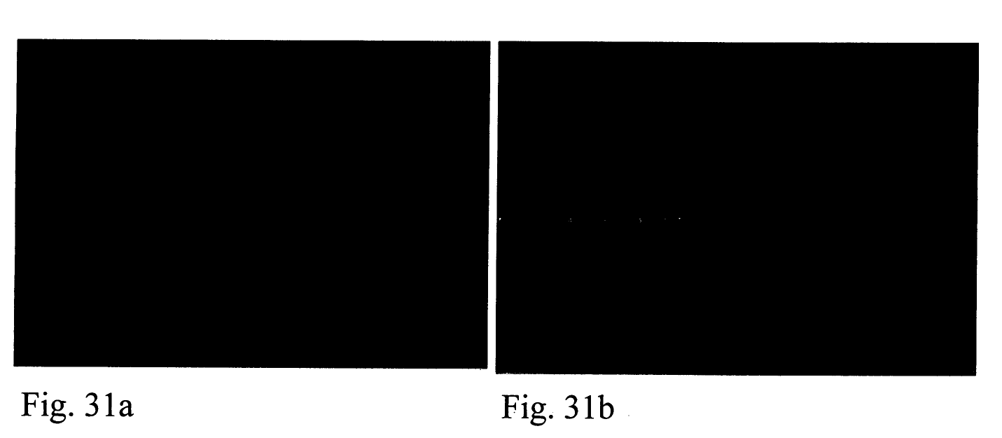

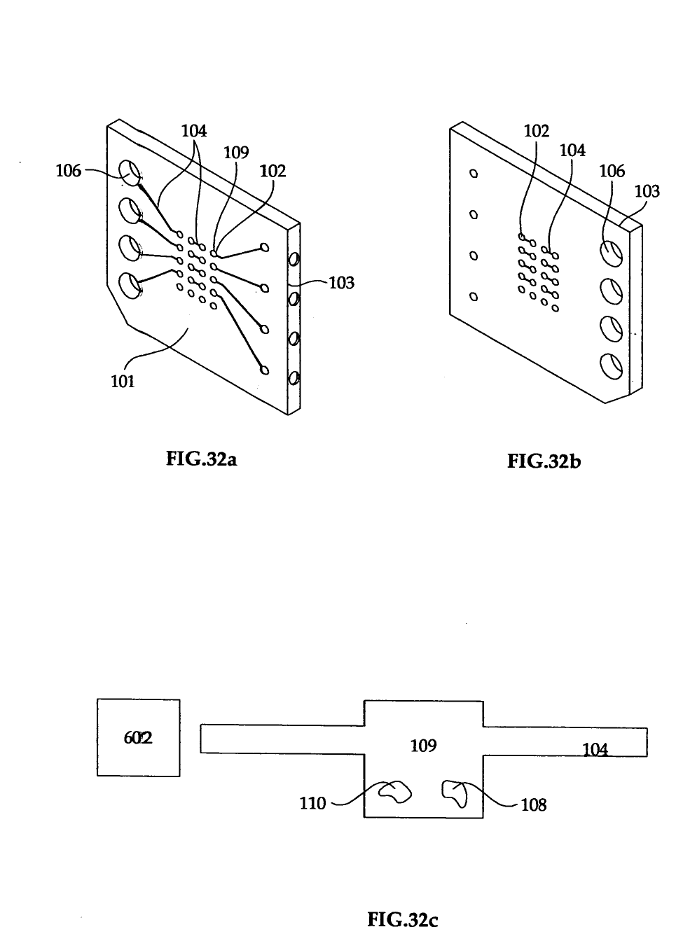

ROS levels in individual sensor cells prior to (FIG. 30a) and following 1 (FIG. 30b) and 10 min (FIG. 30c) of co-incubation with secreting cells are shown. The intracellular level of ROS increased by 21-51% in different individual sensor cells after 1 min of co-incubation, and further increased by 15-32% following 10 min. In control experiment, an increase of 2-15% was found when sensors cells were co incubated under the same conditions with non activated secreting cells. (FIGS. 31a-b)

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

1. An isolated population of cells comprising at least one secretor cell capable of secreting a molecule and at least one sensor cell capable of producing a detectable signal upon being exposed to said molecule.

2. The isolated population of cells of claim 1, wherein said molecule is selected from the group consisting of a small molecule chemical, an ion, a carbohydrate and a polypeptide.

3. The isolated population of cells of claim 2, wherein said small molecule chemical is selected from the group consisting of a reactive oxygen species and a reactive nitrogen species.

4. The isolated population of cells of claim 2, wherein said ion is selected from the group consisting of calcium, magnesium, zink and phosphate.

5. The isolated population of cells of claim 2, wherein said polypeptide is selected from the group consisting of a growth factor, a hormone, a coagulating factor, a cytokine and a chemokine.

6. The isolated population of cells of claim 1, wherein said secretor cell is a cancer cell.

7. The isolated population of cells of claim 1, wherein the population of cells is attached to a support and whereas each of said at least one secretor cell and said at least one sensor cell is attached to said support in an addressable manner.

8. The population of cells if claim 7, wherein said support is configured as a microscope slide.

9. The isolated population of cells of claim 7, wherein said support is configured as a multiwell plate.

10. The isolated population of cells of claim 7, wherein said at least one secretor cell and said at least one sensor cell are in fluid communication therebetween on said support.

11. The isolated population of cells of claim 9, wherein each well of said multiwell plate has a volume between 1×10⁻⁵-1×10⁻¹⁵ μl.

12. The isolated population of cells of claim 1, wherein said at least one secretor cell and said at least one sensor cell are eukaryotic cells.

13. The isolated population of cells of claim 1, wherein said at least one secretor cell and said at least one sensor cell are prokaryotic cells.

14. The isolated population of cells of claim 1, wherein said detectable signal is selected from the group consisting of a morphological signal, a fluorogenic signal and a chromogenic signal.

15. A method of detecting presence, absence or level of a substance in a sample, the method comprising:
(a) exposing at least one secretor cell to the sample, said at least one secretor cell being capable of secreting a molecule when exposed to the substance in the sample;
(b) exposing at least one sensor cell to said molecule, said at least one sensor cell being capable of producing a detectable signal when exposed to said molecule; and
(c) analyzing said detectabb signal to thereby detect presence, absence or level of the substance in the sample.

16. The method of claim 15, wherein said molecule is selected from the group consisting of a small molecule chemical, an ion, a carbohydrate and a polypeptide.

17. The method of claim 16, wherein said small molecule chemical is selected from the group consisting of a reactive oxygen species and a reactive nitrogen species.

18. The method of claim 16, wherein said ion is selected from the group consisting of calcium magnesium, zink and phosphate.

19. The method of claim 16, wherein said polypeptide is selected from the group consisting of a growth factor, a hormone, a coagulating factor, a cytokine and a chemokine.

20. The method of claim 15, wherein said secretor cell is a cancer cell.

21. The method of claim 15, wherein the population of cells is attached to a support and whereas each of said at least one secretor cell and said at least one sensor cell is attached to said support in an addressable manner.

22. The method of claim 21, wherein said support is configured as a microscope slide.

23. The method of claim 21, wherein said support is configured as a multiwell plate.

24. The method of claim 21, wherein said at least one secretor cell and said at leastone sensor cell are in fluid communication therebetween on said support.

25. The method of claim 23, wherein each well of said multiwell plate has a volume between 1×10⁻⁵-10⁻¹⁵ μL.

26. The method of claim 15, wherein said at least one secretor cell and said at least one sensor cell are eukaryotic cells.

27. The method of claim 15, wherein said at least one secretor cell and said at least one sensor cell are prokaryotic cells.

28. The method of claim 15, wherein said detectable signal is selected from the group consisting of a morphological signal, a fluorogenic signal and a chromogenic signal.

29. A method of identifying cells expressing a molecule of interest, the method comprising exposing sensor cells to a plurality of cells potentially capable of secreting the molecule of interest, said sensor cells being capable of producing a detectable signal when exposed to the molecule of interest, thereby identifying the cells expressing the molecule of interest.

30. The method of claim 29, wherein said molecule is selected from the group consisting of a small molecule chemical, an ion, a carbohydrate and a polypeptide.

31. The method of claim 30, wherein said small molecule chemical is selected from the group consisting of a reactive oxygen species and a reactive nitrogen species.

32. The method of claim 30, wherein said ion is selected from the group consisting of calcium, magnesium, zinc and phosphate.

33. The method of claim 30, wherein said polypeptide is selected from the group consisting of a growth factor, a hormone, a coagulating factor, a cytokine and a chemokine.

34. The method of claim 29, wherein said secretor cell is a cancer cell.

35. The method of claim 29, wherein the population of cells is attached to a support and whereas each of said at least one secretor cell and said at least one sensor cell is attached to said support in an addressable manner.

36. The method of claim 35, wherein said support is configured as a microscope slide.

37. The method of claim 35, wherein said support is configured as a multiwell plate.

38. The method of claim 35, wherein said at least one secretor cell and said at least one sensor cell are in fluid communication therebetween on said support.

39. The method of claim 37, wherein each well of said multiwell plate has a volume between 1×10⁻⁵-1×10⁻¹⁵ μl.

40. The method of claim 29, wherein said at least one secretor cell and said at least one sensor cell are eukaryotic cells.

41. The method of claim 29, wherein said at least cne secretor cell and said at least one sensor cell are prokaryotic cells.

42. The method of claim 29, wherein said detectable signal is selected from the group consisting of a morphological signal, a fluorogenic signal and a chromogenic signal.
